(19) United States
(12) Patent Application Publication
(10) Pub. No.: US 2025/0346858 A1
(43) Pub. Date: Nov. 13, 2025

(54) METHODS OF GENERATING THREE-DIMENSIONAL RETINAL ORGANOIDS FROM HUMAN PLURIPOTENT STEM CELLS

(71) Applicant: THE TRUSTEES OF INDIANA UNIVERSITY, Bloomington, IN (US)

(72) Inventors: Jason S. Meyer, Whitestown, IN (US); Jade Harkin, Indianapolis, IN (US)

(21) Appl. No.: 18/862,446

(22) PCT Filed: May 3, 2023
(86) PCT No.: PCT/US2023/020905

§ 371 Date: Nov. 1, 2024

Related U.S. Application Data
(60) Provisional application No. 63/337,906, filed on May 3, 2022.

(51) Int. Cl. C12N 5/079

(52) U.S. Cl. CPC C12N 5/0621; C12N 2500/33; C12N 2501/155; C12N 2506/45; C12N 2513/00

(57) ABSTRACT

Provided herein are methods for directing differentiation of human pluripotent stem cells into retinal organoids with lower variability and higher efficiency than prior art methods.

### CROSS REFERENCE TO RELATED APPLICATIONS

This application claims benefit of U.S. Provisional Application Ser. No. 63/337,906, filed May 3, 2022, the entire contents of which is incorporated by reference herein.

### STATEMENT REGARDING FEDERALLY SPONSORED RESEARCH OR DEVELOPMENT

This invention was made with government support under grants EY024984 and EY033022 awarded by the National Institutes of Health. The government has certain rights in this invention.

### FIELD OF THE INVENTION

Provided herein are methods for directing differentiation of human pluripotent stem cells into three-dimensional retinal organoids. More particularly, provided herein is a refined approach that consistently yields 100% retinal organoid differentiation at much more consistent shape and size, which will be of great utility as a research tool for studies of disease and for pharmacological development.

### BACKGROUND

Human pluripotent stem cells, which include both human embryonic stem cells (hESCs) and human induced pluripotent stem cells (hiPSCs), hold the potential to differentiate into any cell type. As such, they can serve as comprehensive model systems of human cell genesis, particularly at early developmental stages that would otherwise be inaccessible to investigation. In addition, patient-derived hiPSC lines have a unique capacity to model human disease, although the scope of disorders amenable to this form of study is limited. Major considerations when creating hiPSC disease models include the capacity to efficiently generate, identify and isolate relevant cell populations, as well as recapitulate and assay critical aspects of the disease mechanism.

Retinal cell types are particularly well-suited for the investigation of cell development and dysfunction using pluripotent stem cell technology. The vertebrate retina harbors a modest repertoire of major cell classes sequentially produced via a conserved series of events. Furthermore, the effects of inherited and acquired retinal degenerative diseases (RDD) are often limited initially to a specific cell class, which simplifies the study of cellular mechanisms that incite RDD and the evaluation of potential therapies.

Previous studies have demonstrated the ability of human pluripotent stem cells to differentiate along the retinal lineage with varying efficiencies, with one protocol achieving a near uniform retinal cell fate using the WA01 hESC line (Lamba et al., 2011). However, pluripotent stem cell-derived retinal cells, particularly those from hiPSCs, are most often found in mixed populations that include some non-retinal or unidentified cell types. Further complicating matters is the fact that several markers used for retinal cell identification (e.g., calretinin, PKC.alpha., Tuj1) also label cells found in other regions of the CNS. As such, a means to isolate developmentally synchronized populations of multipotent retinal progenitor cells (RPCs) across multiple hESC and hiPSC lines would be desirable. The RPCs and their definitive retinal progeny could then be used to study mechanisms of human retinal development and disease, examine retinal cell function, and devise and test RDD treatments.

In recent years, several groups have described the ability to direct human pluripotent stem cells (hPSCs) to a retinal fate (Lamba et al., 2006, 2010; Osakada et al., 2008; Carr et al., 2009; Hirami et al., 2009; Nakano et al., 2012; Buchholz et al., 2013). In order to serve as an effective in vitro model for human retinogenesis, as well as provide a foundation for translational applications, the stepwise differentiation of hPSCs through all of the major stages of retinogenesis helps to ensure the proper differentiation and prospective identification of hPSC-derived retinal progeny (Meyer et al., 2009, 2011; Gamm and Meyer, 2010; Sridhar et al., 2013).

Methods to differentiate human pluripotent stem cells to RPCs, retinal pigment epithelium (RPE), and photoreceptor-like cells in a manner that mimicked normal human retinogenesis have been previously described (Meyer et al., 2009). Methods wherein transient morphological features were used to isolate structures with characteristics reminiscent of the optic vesicle have (OV) have also been described. (Meyer et al., 2011). Using such OV-like structures, it was possible to study principles of early human retinal development, monitor the sequence and timing of neuroretinal cell genesis, and optimize RPC and RPE production efficiencies in recalcitrant hiPSC lines.

### SUMMARY

Disclosed herein are methods for directing differentiation of human pluripotent stem cells into three-dimensional retinal organoids. More particularly, provided herein is a refined approach that consistently yields 100% retinal organoid differentiation at much more consistent shape and size, which will be of great utility as a research tool for studies of disease and for pharmacological development.

One aspect for a method of preparing PSCs to for use in retinal organoid differentiation is provided. Another aspect for a method of preparing aggregates for use in differentiating three-dimensional retinal organoid tissue is provided. A further aspect for methods of forming three-dimensional retinal organoid tissue, as well as differentiating PSC aggregates to three-dimensional retinal organoid tissue, are provided. In embodiments, the methods comprise one or more of the following steps:
(aa) contact the PSCs with accutase and a Rock inhibitor;
(bb) dissociate the PSCs to single cells
(cc) culture the dissociated cells in mTeSR for at least one day;
(dd) culture the of (c) in (1) mTeSR, and (2) Neural induction medium, at a ratio of 3:1, for one day.
a) day 1: culture cell aggregates in (1) mTeSR, and (2) Neural induction medium, at a ratio of 1:1, for one day;
b) day 2: culture the aggregates of (a) in (1) mTeSR and (2) Neural induction medium, at a ratio of 1:3 for 1 day;
c) day 3: culture the aggregates of (b) in Neural induction medium for three days;
d) day 6: culture the aggregates of (c) in Neural induction medium and BMP4 for two days;
e) day 8: transfer 10-20 aggregates of (d) to a single well of a 6-well culture plate and culture the aggregates and culture in the presence of reduced concentrations of BMP4 for eight days;
f) day 15: replace all media and culture aggregates in NIM, in the absence of FBS;
g) day 16: culture the aggregates of (f) in RDM supplemented with 1% FBS for six days;
h) day 22; culture the aggregates of (g) in RDM with 10% FBS and 1× Glutamax for three days;
i) day 25: culture the aggregates of (h) in RDM with 10% FBS, 1× Glutamax, and taurine for 25 days;
j) day 50: culture the aggregates of (i) in RDM, 10% FBS, 1× Glutamax, taurine, retinoic acid, DHA, and BDNF, for 40 days;
k) day 90; culture aggregates of (j) in RDM, 10% FBS, 1× Glutamax, taurine, DHA, and BDNF.

### BRIEF DESCRIPTION OF THE DRAWINGS

The embodiments set forth in the drawings are illustrative and exemplary in nature and not intended to limit the subject matter defined by the claims. The detailed description of the illustrative embodiments can be understood when read in conjunction with the following drawings.

FIGS. 1A-1E illustrate traditional versus standardized differentiation methods for human pluripotent stem cells (hPSCs) to form embryoid bodies. Traditional differentiation methods use dispase to dissociate the human pluripotent stem cells (hPSCs) to form embryoid bodies (FIG. 1A), which leads to cellular aggregates that are variable in both their sizes and shapes (as seen in the Day 6 images on right). Standardized differentiation methods use accutase to dissociate the hPSCs into single cells and then cells can be added to 96 U bottom low adhesion plates allowing control of both the size and shape of the cellular aggregates (FIG. 1A). Using the standardized method, hPSCs were plated at different densities (ranging from 250 cells per well to 8000 cells per well), and then the size and circularity of the aggregates quantified at Day 3 (FIGS. 1B-1C), and Day 6 (FIGS. 1D-1E), compared to the traditional method of differentiation (FIGS. 1C and 1E). As seen in the figures both the size and circularity of the aggregates at the early stages of differentiation are more consistent (FIGS. 1B, 1D and 1F). Non-hPSC lines, ES and iPS cell lines, show that this method is highly reproducible and consistent across multiple cell lines (FIGS. 1C and 1E).

FIGS. 2A-20 demonstrate enriched retinal organoid production using the standardized differentiation method. Using the IMR90.4 cell line that has a SIX6-GFP reporter, hPSCs were differentiated using both the traditional and standardized methods until 25 Days of total differentiation, with generated aggregates being treated with BMP4 at Day 6. Untreated organoids, BMP4 treated organoids, and BMP4 treated organoids were compared for BMP4 treatment, and additionally with LDN (a small molecule BMP4 inhibitor) treatment by both traditional and standardized differentiation methods. Untreated using the traditional method (FIG. 2A), approx. 30% of organoids express GFP (FIGS. 2B and 2K), and after treatment with BMP4 (FIGS. 2C and 2K), about 80%-90% of the organoids express GFP (FIGS. 2D and 2K). Using the standardized method, after treatment with BMP4 (FIG. 2E), 100% of the organoids become retinal, as shown by the GFP expression (FIGS. 2F and 2K). Conversely, when BMP4 signaling was blocked using LDN, organoids differentiated using both the traditional (FIG. 2G) and standardized method (FIG. 2I) do not express GFP (FIGS. 2H, 2I, and 2K). Additionally, both the size and shape of the organoids were quantified at Day 25 (FIGS. 2C, 2L, and 2M). The organoids differentiated using the standardized method of differentiation are more consistent in their size and circularity at Day 25 when compared to organoids differentiated using the traditional methods of differentiation (FIGS. 2M-2N). Initial cell seeding density at 250, 500, 1000, 2000, 4000, and 8000 cells per well shows improved aggregate efficiency and quality of cell aggregate for differentiation; at least 2000 cell seeds optimal minimum to achieve 100% efficiency in retinal organoid differentiation. (FIG. 2 O).

FIG. 3 demonstrates the standardized differentiation method for retinal organoids reproducibility across multiple stem cell lines. Organoids from six different ES and iPS cell lines, H7 ES, JM2019 iPS, PGP1 iPS, IMR90-4 iPS, H9 ES, and WTC11 iPS, were differentiated and stained for the retinal progenitor marker Chx10. All sections stain positive for the retinal marker Chx10 (shown in red).

FIGS. 4A-4D illustrate the transcriptome of early stages of differentiation of early cell fate determination events after treatment with either BMP4 or LDN at Day 6. RNA from cell aggregates was collected at Day 6 before any treatments, then treated at day 8 with either BMP4 or LDN, were confirmed at Day 25 that the organoids expressed GFP (FIG. 4A). mRNA-seq data comparing: (1) Day 8 BMP4 with Day 6 untreated (FIG. 4B); (2) Day 8 LDN with Day 6 untreated (FIG. 4C); and (3) Day 8 LDN with Day 8 LDN (FIG. 4D). Comparing Day 8 BMP4 with Day 6 untreated, after only 2 days treatment with BMP4 there is an upregulation in the expression of retinal specific genes including SIX6, RAX, LHX9 VSX2 (CHX10), PAX6 (FIG. 4B). Conversely, Day 8 LDN compared with Day 6 untreated, there is an upregulation of more cortical/forebrain related genes including FOXG1 and MAP2 (FIG. 4C). For Day 8 BMP4 compared with Day 8 LDN, there is an increase in retinal specific genes including VSX2 (CHX10), SIX6, LHX9 and RAX, with a decrease in more cortical/forebrain related genes including FOXG1 and MAP2 (FIG. 4D).

FIGS. 5A-5X demonstrate enhanced retinal ganglion cell (RGC) differentiation to organoid stages by expression in retinal neurons. Retinal organoids differentiated using both traditional and standardized methods using a Brn3b-GFP reporter cell line to examine the first born retinal neurons, the RGCs. Using qPCR and mean fluorescent intensity quantifications of the RGC specific marker Brn3b after 30 days of differentiation (FIGS. 5A, 5B), the standardized retinal organoids robustly express Brn3b positive RGCs (FIGS. 5D, 5H), while organoids differentiated using traditional methods have very low levels of Brn3b expression at Day 30 (FIGS. 5C, 5G). Retinal progenitor marker Chx10 staining shows RGCs develop faster when organoids are differentiated using the standardized method (FIG. 5F) compared to the traditional method (FIG. 5E). Using qPCR and mean fluorescent intensity quantifications of the CRX after 60 days of differentiation, the standardized retinal organoids are robustly expressing CRX (FIGS. 5L, 5P), while organoids differentiated using traditional methods have low levels of CRX expression at Day 60 (FIGS. 5K, 5O). Retinal progenitor marker Chx10 staining shows nerve cells develop faster when organoids are differentiated using the standardized method (FIG. 5N) compared to the traditional method (FIG. 5M). Retinal organoid tissue differentiated using both traditional and standardized methods after 150 day of differentiation and formation (FIGS. 5Q, 5R) using a NRL marker, the cells are robustly expressing NRL positive (FIG. 5T), compared to traditional differentiated methods (FIG. 5S). NRL/ARR3/CRX staining shows differentiated retinal cell development using the standardized method (FIG. 5V) compared to the traditional method (FIG. 5U). NRL regulation of rhodopsin is also enhanced using standardized methods (FIG. 5X) compared to traditional methods (FIG. 5W).

FIG. 6 demonstrates expedited photoreceptor differentiation at later stages of retinal organoids differentiation for expression of photoreceptors. Organoids were differentiated for 70 days using both the traditional and standardized methods and then analyzed for the expression of the photoreceptor specific marker CRX. Photoreceptor differentiation is expedited when retinal organoids are differentiated using standardized methods compared to traditional methods.

### DETAILED DESCRIPTION

In the following detailed description, reference is made to the accompanying drawings that form a part hereof. The embodiments are described in sufficient detail to enable those skilled in the art to practice the invention, and it is understood that other embodiments may be utilized and that changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined by the appended claims.

Unless defined otherwise, all technical and scientific terms used in this disclosure with the appended claims have the same meaning that is commonly understood by one of ordinary skill in art to which the subject matter pertains. Although any methods and materials similar or equivalent to those described herein can be used in the practice for testing of the present invention, the preferred materials and methods are described herein. As used in the specification and the appended claims, unless specified to the contrary, the following terms have the meaning indicated to facilitate the understanding of the disclosure.

### Terminology

The indefinite articles “a” and “an,” as used herein in the specification and in the claims, unless clearly indicated to the contrary, should be understood to mean “at least one.”

A range includes each individual member. Thus, for example, a group having 1-3 members refers to groups having 1, 2, or 3 members.

As used herein, “about” means within 10% of a stated concentration range, density, temperature, time frame, etc.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

As used herein, “a medium consisting essentially of” means a medium that contains the specified ingredients and those that do not materially affect its basic characteristics.

As used herein, “effective amount” means an amount of an agent sufficient to evoke a specified cellular effect according to the present invention.

As used herein, the term “stem cell” refers to cells that are undifferentiated or partially differentiated cells that can differentiate into various types of cells and proliferate indefinitely to produce more of the same stem cell. They are the earliest type of cell in a cell lineage. Stem cells are found in both embryonic and adult organisms, but may have slightly different properties in each. They are usually distinguished from progenitor cells, which cannot divide indefinitely, and precursor or blast cells, which are usually committed to differentiating into one cell type.

As used herein, the term “pluripotent” stem cell refers to a cell that is not capable of growing into an entire organism, but is capable of giving rise to cell types originating from all three germ layers, i.e., mesoderm, endoderm, and ectoderm, and may be capable of giving rise to all cell types of an organism. Pluripotency can be a feature of the cell per see, e.g. in certain stem cells, or it can be induced artificially. Examples of pluripotent stem cells include, but are not limited to, embryonic stem cells (ES), embryonic stem cells derived from a cloned embryo obtained by nuclear transplantation (ntES), spermatogonial stem cells (“GS cells”), embryonic germ cells (“EG cells”), induced pluripotent stem cells (iPS) and multipotent cells derived from cultured fibroblasts. In some embodiments, ES cells and/or iPS cells such as human ES and/or iPS cells are used in the methods and compositions disclosed herein.

As used herein, the term “embryonic stem cell” refers to cells that are totipotent and derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. These cells express Oct-4, SSEA-3, SSEA-4, TRA-1-60 and TRA-1-81, and appear as compact colonies having a high nucleus to cytoplasm ratio and prominent nucleolus. ESCs are commercially available from sources such as WiCell Research Institute (Madison, Wis.). Embryonic stem cells can also be obtained directly from suitable tissue, including, but not limited to human tissue, or from established embryonic cell lines. In one embodiment, embryonic stem cells are obtained as described by Thomson et al. (U.S. Pat. Nos. 5,843,780 and 6,200,806; Science 282:1145, 1998; Curr. Top. Dev. Biol. 38:133 ff, 1998; Proc. Natl. Acad. Sci. U.S.A. 92:7844, 1995).

As used herein, the terms “induced pluripotent stem cell” or “iPSC”, which are used interchangeably herein, refer to pluripotent cells derived from differentiated cells. For example, iPSCs can be obtained by overexpression of transcription factors such as Oct4, Sox2, c-Myc and Klf4 according to the methods described in Takahashi et al. (Cell, 126:663-676, 2006). Other methods for producing iPSCs are described, for example, in Takahashi et al. Cell, 131:861-872, 2007 and Nakagawa et al. Nat. Biotechnol. 26:101-106, 2008. Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

“Assembloids” refer to self-organizing three-dimensional miniature organs grown in vitro made by combining two or more organoids resembling distinct areas that can be used to model aspects of interactions that occur in a subject

“Organoid” refers to a tiny, self-organized three-dimensional multicellular in vitro tissue construct that mimics aspects of its corresponding in vivo organ, such that it can be used to study aspects of that organ in the tissue culture dish. An organoid is derived from stem cells and it can be crafted to replicate much of the complexity of an organ, or to express selected aspects of it like producing only certain types of cells.

As used herein, a cellular “aggregate” or “aggregates” refer to clusters of cells, typically cells of the same type, such as pluripotent stem cells, that are loosely grouped together. “Aggregation” refers to the clustering together and adhesion of initially separated cells to form an aggregate.

“Retinal organoids (ROs)” refer to three-dimensional structures derived from pluripotent stem cells (e.g., human PSCs) which recapitulate aspects of the spatial and temporal differentiation of the retina, and which may serve, for example, as effective in vitro models of retinal development.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be susceptible to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. The subject therapy will desirably be administered during the symptomatic stage of the disease, and in some cases after the symptomatic stage of the disease.

### DESCRIPTION

Pluripotent stem cells (PSCs), for example human PSCs (hPSCs), possess the unique ability to readily differentiate into any cell type of the body. As such, they can serve as comprehensive and novel tools for drug screening, disease modeling, and cell replacement therapies. Although previous studies have demonstrated the ability to differentiate hPSCs to a retinal lineage, consistency and reproducibility of differentiation products has been lacking.

Retinal organoids can be differentiated from PSCs that effectively recapitulate the major stages of retinogenesis. These organoids are becoming valuable tools for studying retinogenesis, retinal diseases, disease progress, to screen compounds for potential therapeutic efficacy, and even provide a source of replacement cells for transplantation purposes. Yet shortcomings in the efficiency and reproducibility of current retinal organoid differentiation protocols have hindered their ability to serve as effective models for the earliest stages of retinal lineage specification.

The present disclosure provides a novel retinal organoid differentiation protocol using more standardized, rapid reaggregation methods to generate highly reproducible retinal organoids from PSCs, including hPSCs. Bone morphogenetic proteins (BMPs) are a group proteins within the transforming growth factor beta (TGFβ) superfamily that bind to cell surface receptors, and include growth and differentiation factors. BMP signaling contributing to retinal specification was analyzed by treatment with either BMP4 protein or the BMP inhibitor LDN-193189, and differentiation efficiency was assessed at various time points based on morphological analyses and the expression of retinal markers. Additionally, to identify transcriptional changes that underly retinal fate determination events, mRNA-seq analyses were conducted at the earliest stages of retinal specification.

As shown herein, retinal organoids generated using quick reaggregation methods were highly reproducible in both their size and shape compared to more traditional methods. Following treatment of early aggregates with either BMP4 or LDN-193189, pure populations of either retinal or forebrain organoids were derived, respectively. Subsequently, RNA-seq methods analyzed the transcriptional profile of the earlies stages of retinal vs forebrain specification, long before these lineages have been reliably identified previously. These refined methods also yielded retinal organoids with greatly expedited differentiation timelines, with differentiated retinal neurons arising at earlier stages than traditional differentiation methods, also exhibiting higher levels of self-organization.

Thus, one aspect is methods of differentiating human pluripotent stem cells under conditions that promote differentiation of the pluripotent stem cells into three-dimensional retinal tissue. Generally, cells of retinal tissue are identified by their surface phenotype, by the ability to respond to growth factors, and being able to differentiate in vivo or in vitro into particular cell lineages.

Differentiation protocols are well known to those of ordinary skill in the art. For example, some of the traditional differentiation protocols are disclosed in U.S. Pat. No. 9,752,119. The disclosed methods are standardized to improve differentiating human pluripotent stem cells under conditions, which promote efficient differentiation. In some embodiments, to form aggregates, a confluent culture of pluripotent stem cells can be chemically, enzymatically or mechanically dissociated from a surface, such as Matrigel® into clumps, aggregates, or single cells. In exemplary embodiments, the dissociated cells (as clumps, aggregates, or single cells) are plated onto a surface in a protein-free basal medium such as Dulbecco's Modified Eagle's Medium (DMEM)/F12, mTeSR™ (StemCell Technologies; Vancouver, British Columbia, Canada), and TeSR™. The full constituents and methods of use of TeSR™ are described in Ludwig et al. See, e.g., Ludwig T, et al., “Feeder-independent culture of human embryonic stem cells,” Nat. Methods 3:637-646 (2006); and Ludwig T, et al., “Derivation of human embryonic stem cells in defined conditions,” Nat. Biotechnol. 24:185-187 (2006). Other DMEM formulations suitable for use herein include, e.g., X-Vivo (BioWhittaker, Walkersville, MD) and StemPro® (Invitrogen; Carlsbad, CA).

In some embodiments, aggregates of pluripotent stem cells are cultured in the presence of a kinase inhibitor, such as a Rho kinase (ROCK) inhibitor. Kinase inhibitors, such as ROCK inhibitors, are known to protect single cells and small aggregates of cells. See, e.g., US Patent Application Publication No. 2008/0171385, and Watanabe K, et al., “A ROCK inhibitor permits survival of dissociated human embryonic stem cells,” Nat. Biotechnol. 25:681-686 (2007). ROCK inhibitors are shown below to significantly increase pluripotent cell survival on chemically defined surfaces. ROCK inhibitors suitable for use in the disclosed methods include, but are not limited to, (S)-(+)-2-methyl-1-[(4-methyl-5-isoquinolinyl)sulfonyl]homopiperazine dihydrochloride (informal name: H-1152), 1-(5-isoquinolinesulfonyl) piperazine hydrochloride (informal name: HA-100), 1-(5-isoquinolinesulfonyl)-2-methylpiperazine (informal name: H-7), 1-(5-isoquinolinesulfonyl)-3-methylpiperazine (informal name: iso H-7), N-2-(methylamino)ethyl-5-isoquinoline-sulfonamide dihydrochloride (informal name: H-8), N-(2-aminoethyl)-5-isoquinolinesulphonamide dihydrochloride (informal name: H-9), N-[2-p-bromo-cinnamylamino)ethyl]-5-isoquinolinesulfonamide dihydrochloride (informal name: H-89), N-(2-guanidinoethyl)-5-isoquinolinesulfonamide hydrochloride (informal name: HA-1004), 1-(5-isoquinolinesulfonyl) homopiperazine dihydrochloride (informal name: HA-1077), (S)-(+)-2-Methyl-4-glycyl-1-(4-methylisoquinolinyl-5-sulfonyl) homopiperazine dihydrochloride (informal name: glycyl H-1152) and (+)-(R)-trans-4-(1-aminoethyl)-N-(4-pyridyl)cyclohexanecarboxamide dihydrochloride (informal name: Y-27632).

The kinase inhibitor can be provided at a concentration sufficiently high that the cells survive and remain attached to the surface. An inhibitor concentration between about 3 μM to about 10 μM can be suitable, preferably about 3 μM, about 4 μM, about 5 μM, about 6 μM, about 7 μM, about 8 μM, about 9 μM, or about 10 μM. At lower concentrations, or when no ROCK inhibitor is provided, undifferentiated cells typically detach, while differentiated cells remain attached to the defined surface.

Fibroblast Growth Factor 2 (FGF-2) is an agonist of FGF signaling, and FGF signaling can be antagonized using, for example, the small molecule inhibitor PD-173074. BMP4 is an agonist of BMP signaling, and BMP signaling can be antagonized using, for example, the small molecule inhibitor LDN-193189. TGFβ-1 and Activin A are agonists of TGFβ signaling, and TGFβ signaling can be antagonized using, for example, the small molecule inhibitor SB-431542. CHIR-99021 is an agonist of the Wnt/β-catenin signaling pathway, and Wnt/β-catenin signaling can be antagonized using, for example, the small molecule inhibitor XAV-939. Other Wnt agonists include inhibitors/antagonists of the molecule Glycogen Synthase Kinase 3 (GSK3).

In some embodiments, a semi-solid composition of extracellular matrix proteins may be used for growth and differentiation of pluripotent stem cells. An example of semi-solid composition of extracellular matrix proteins is a commercially available product Geltrex® basement membrane matrix. Geltrex® basement membrane matrix is suitable for use with human pluripotent stem cell applications using StemPro® hESC SFM or Essential 8™ media systems. In some embodiments, the semi-solid composition comprises two or more extra cellular matrix proteins such as, for example, laminin, entactin, vitronectin, fibronectin, a collagen, or combinations thereof.

In some embodiments, pluripotent stem cells, particularly hPSCs, are cultured in a chemically-defined basal culture medium formulation comprising the defined components of culture medium “DF3S” as set forth in Chen et al., Nature Methods 8:424-429 (2011). As used herein, the terms “E7 culture medium” and “E7” are used interchangeably and refer to a chemically defined culture medium comprising or consisting of DF3S supplemented to further comprise insulin (20 μg/mL), transferrin (10.67 ng/ml) and human Fibroblast Growth Factor 2 (FGF2) (100 ng/mL). As used herein, the terms “E8 culture medium” and “E8” are used interchangeably and refer to a chemically defined culture medium comprising or consisting of DF3S supplemented by the addition of insulin, transferrin, human FGF2, and human Transforming Growth Factor Beta 1 (TGFβ1). Exemplary supplements may be included with insulin (20 μg/mL), transferrin (10.67 ng/mL), human FGF2 (100 ng/ml), and TGFβ1 (1.75 ng/ml).

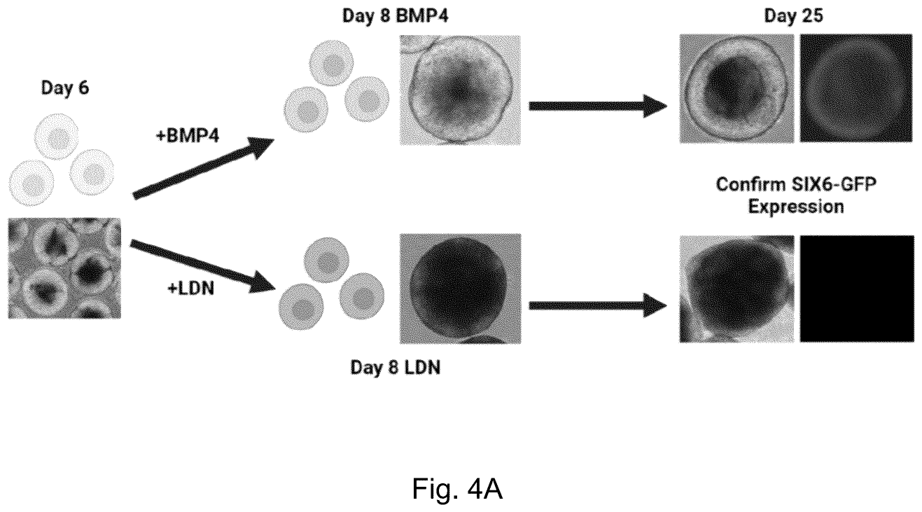

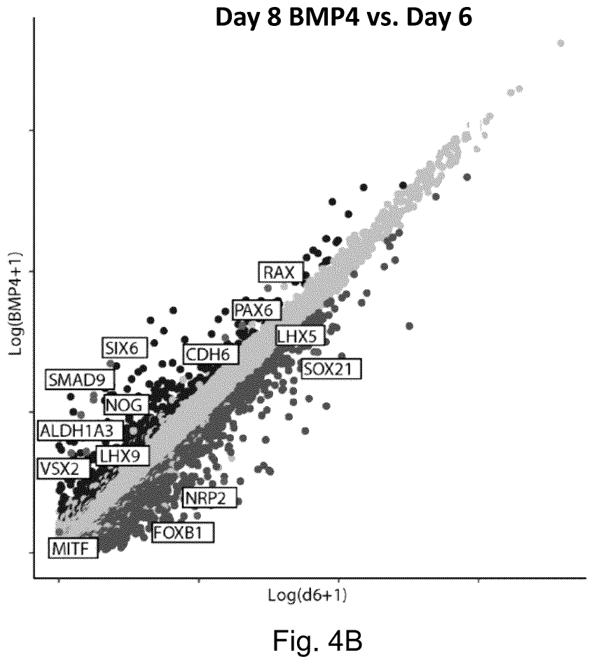

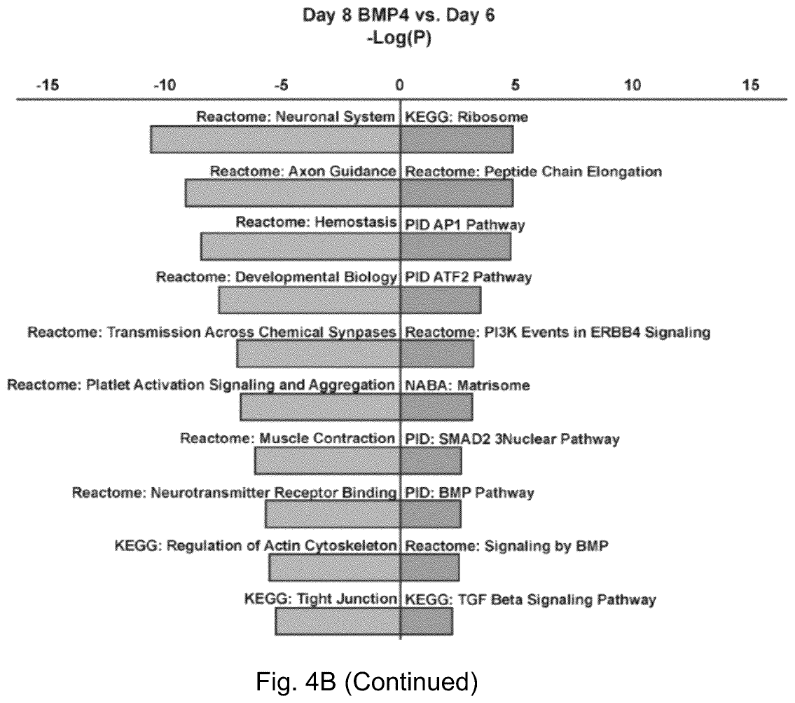

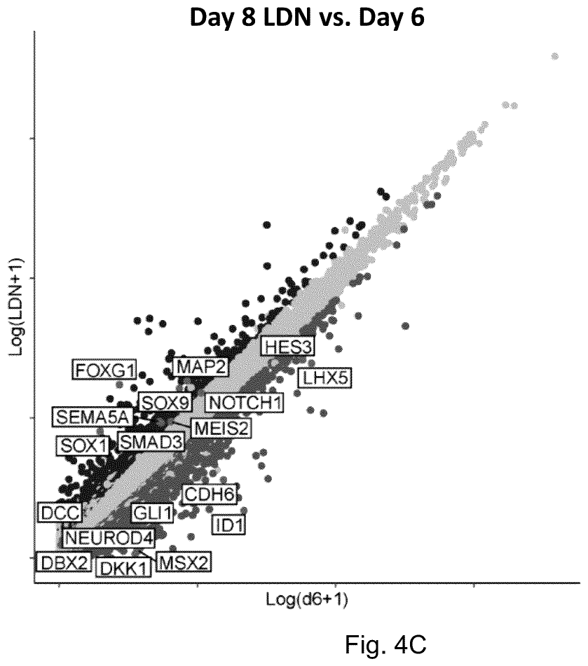

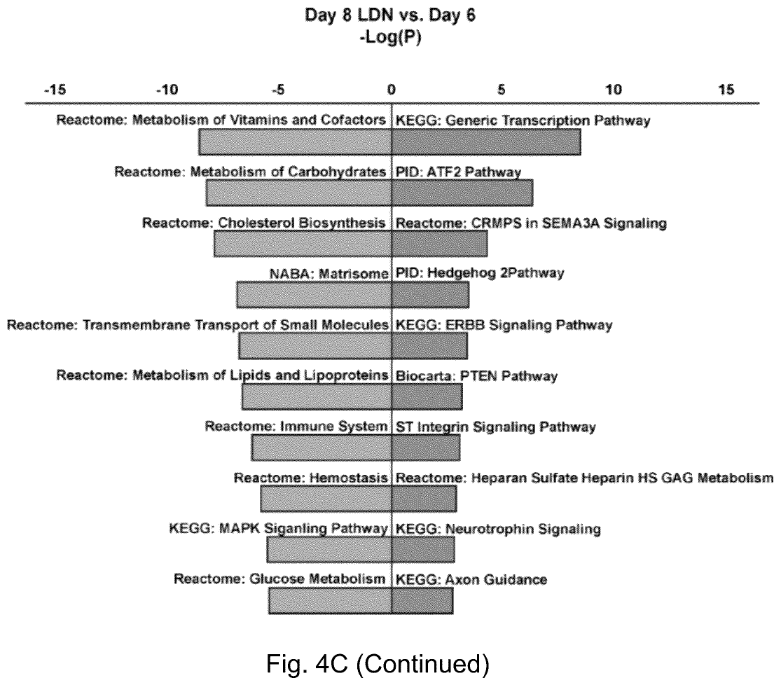

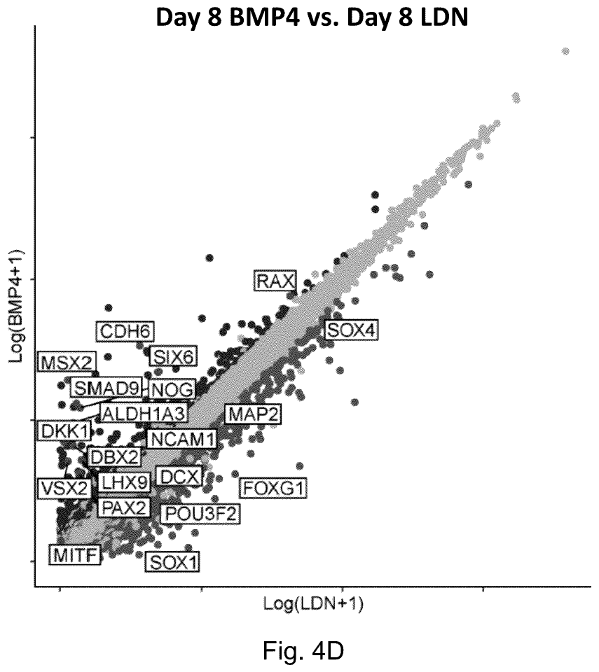

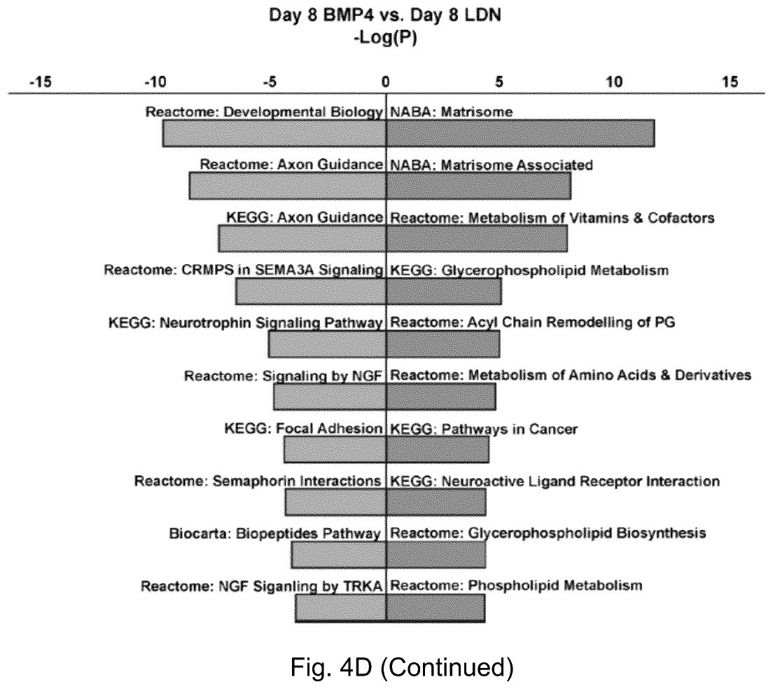

Any appropriate method can be used to detect expression of biological markers characteristic of cell types used in the disclosed embodiments. For example, the presence or absence of one or more biological markers can be detected using, for example, RNA sequencing methods (RNA-seq), immunohistochemistry, polymerase chain reaction, qRT-PCR, or other technique that detects or measures gene expression. In exemplary embodiments, a cell population obtained according to a method provided herein is evaluated for expression (or the absence thereof) of biological markers of retinal tissue such as those listed in FIG. 4B-4D, including but not limited to Brn3b (POU4F2 (POU class 4 transcription factor 2)), visual system homeobox 2 (VSX2 or CHX10), SIX6 (sine oculis 6), RAX (Retina and Anterior Neural Fold Homeobox), LHX9 (LIM homeobox 9), and PAX6 (Paired-box 6), and cortical/forebrain related genes including FOXG1 (Forkhead box G1) and MAP2 (microtubule-associated protein 2) (FIG. 4B-4D). Quantitative methods for evaluating expression of markers at the protein level in cell populations are also known in the art. For example, flow cytometry is used to determine the fraction of cells in a given cell population that express or do not express biological markers of interest. Differentiated cell identity is also associated with downregulation of pluripotency markers such as NANOG and OCT4 (relative to human ES cells or induced pluripotent stem cells).

Induced pluripotent stem (iPS) cells can be used according to the presently disclosed methods. Human iPS cells can be used to obtain primitive macrophages and microglial cells having the genetic complement of a particular human subject. For example, it may be advantageous to obtain retinal cells that exhibit one or more specific phenotypes associated with or resulting from a particular disease or disorder of the particular mammalian subject. In such cases, iPS cells are obtained by reprogramming a somatic cell of a particular subject according to methods known in the art. See, for example, Yu et al., Science 324 (5928): 797-801 (2009); Chen et al., Nat. Methods 8 (5): 424-9 (2011); Ebert et al., Nature 457 (7227): 277-80 (2009); Howden et al., Proc. Natl. Acad. Sci. U.S.A 108 (16): 6537-42 (2011). Induced pluripotent stem cell-derived retinal tissues can be used to screen drug candidates in tissue constructs that recapitulate retinal tissue in an individual having, for example, a particular disease. Subject-specific somatic cells for reprogramming into induced pluripotent stem cells can be obtained or isolated from a target tissue of interest by biopsy or other tissue sampling methods. In some cases, subject-specific cells are manipulated in vitro prior to use in a three-dimensional tissue construct of the invention. For example, subject-specific cells can be expanded, differentiated, genetically modified, contacted to polypeptides, nucleic acids, or other factors, cryo-preserved, or otherwise modified prior to introduction to a three-dimensional tissue construct.

In some embodiments, human pluripotent stem cells (e.g., human ESCs or iPS cells) are cultured in the absence of a feeder layer (e.g., a fibroblast layer), a conditioned medium, or a culture medium comprising poorly defined or undefined components. As used herein, the terms “chemically defined medium” and “chemically defined cultured medium” also refer to a culture medium containing formulations of fully disclosed or identifiable ingredients, the precise quantities of which are known or identifiable and can be controlled individually. As such, a culture medium is not chemically defined if (1) the chemical and structural identity of all medium ingredients is not known, (2) the medium contains unknown quantities of any ingredients, or (3) both. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions. As used herein, the term “serum-free” refers to cell culture materials that are free of serum obtained from animal (e.g., fetal bovine) blood. In general, culturing cells or tissues in the absence of animal-derived materials (i.e., under xenogen-free conditions) reduces or eliminates the potential for cross-species viral or prion transmission.

### Methods

In some embodiments, methods directing differentiation of pluripotent stem cells into three-dimensional retinal organoids are provided that result in organoids of more consistent shape and size, and the methods are more efficient than traditional (prior art) methods. In some embodiments, the methods comprise those disclosed in Example 1. Background may be found in WO2021081069 (PCT/US2020/056624) and U.S. Pat. No. 10,280,400.

a. Exemplary Non-Limiting Methods

Maintenance and expansion of hPSCs. Different lines of hPSCs were utilized in this study, including those with or without an RGC-specific fluorescent reporter. hPSCs were initially maintained in an undifferentiated state as previously described (see e.g., Ohlemacher, S. K., Iglesias, C. L., Sridhar, A, Gamm, D. M. & Meyer, J. S. Generation of highly enriched populations of optic vesicle-like retinal cells from human pluripotent stem cells. CURRENT PROTOCOLS IN STEM CELL BIOLOGY 32, 1h.8.1-20; see also Fligor C M, Huang K C, Lavekar S S, VanderWall K B, Meyer J S (2020), Differentiation of retinal organoids from human pluripotent stem cells, METHODS CELL BIOL 159:279-302). Briefly, cells were maintained in mTeSR1 medium on a Matrigel substrate. Upon reaching approximately 70% confluency, cells were mechanically passaged with dispase (2 mg/ml) and split at a ratio of 1:6, with passaging of cells occurring every 4-5 days.

Differentiation of organoids from hPSCs. For retinal organoids, hPSCs were differentiated to a retinal lineage following previously established protocols (Fligor et al. 2020). Briefly, embryoid bodies (EB) were generated by lifting hPSCs from Matrigel-coated wells using dispase (2 mg/mL). EBs were maintained in suspension and gradually transitioned to a chemically defined neural induction medium (NIM), which consisted of DMEM/F12 (1:1), N2 supplement, MEM non-essential amino acids, heparin (2 μg/mL) and PSA. After 6 days, 1.5 nM of BMP4 was added to encourage retinal lineage differentiation. After 8 days, the EBs were plated onto 6-well plates with 10% FBS to ensure adhesion. Half media changes were performed on days 9 and 12 with a full media change occurring on day 15. After 16 days of differentiation, cell aggregates were mechanically lifted and kept in suspension in Retinal Differentiation Medium (RDM), which consisted of DMEM/F12 (3:1), B27 supplement, MEM non-essential amino acids, and PSA. Retinal organoids containing presumptive RGCs were maintained in this medium until experimental time points indicated.

Immunocytochemistry and Imaging. For cryostat sectioning, retinal organoids were fixed with 4% paraformaldehyde, washed 3× in PBS, and then equilibrated in a 20% and then 30% sucrose solution overnight at 4° C. Once reaching equilibrium, organoids were embedded in OCT and frozen on dry ice and sections were cut at 11 μm thickness. Similarly, RGCs grown on coverslips were fixed in 4% paraformaldehyde and washed 3× in PBS before staining. Immunocytochemical staining of samples was performed as follows. Briefly, permeabilization was performed in 0.2% Triton X-100 for 10 minutes and samples were then blocked in 10% donkey serum for one hour at room temperature. Primary antibodies were diluted in 0.1% Triton X-100 and 5% donkey serum and applied overnight at 4° C. The following day, samples were washed in PBS and blocked with 10% donkey serum for IO minutes. Secondary antibodies were diluted 1:1000 in 0.1% Triton X-100 and 5% donkey serum and applied for one hour at room temperature. Finally, cells were washed with PBS and mounted onto slides for imaging.

Quantification and statistical analysis. The number of cells expressing unique retinal markers was quantified in cryostat sections of retinal organoids at indicated timepoints. Multiple biological replicates were obtained at each time point (n=3) and Image-J was used to quantify the expression of each marker as indicated in results. One-Way ANOVA statistical analyses at 95% confidence (post hoc Tukey) was performed, excluding outliers, to determine significant differences in cell counts over time. Statistical significances were determined based on a p value less than 0.05. To analyze retinal organoid-derived RGCs, mCherry- or tdTomato positive RGCs were quantified, and the co-expression of these reporters with RGC or other retinal cell type markers was quantified using the Image-J cell counter. Four distinct regions of at least three coverslips were imaged and quantified, with these experiments repeated with at least three different groups of cells. The percentage of mCherry-positive cells colocalizing with retinal cell type markers and the standard error of the mean was quantified.

b. Basic Protocols 1˜4 (See U.S. Pat. No. 10,280,400)
Basic Protocol 1 Enzymatic Passaging of hPSCs

The following procedure is used to maintain and passage hPSCs for long-term use (Thomson et al., 1998; Ludwig et al., 2006; Takahashi et al., 2007; Yu et al., 2007; Park et al., 2008; Meyer et al., 2009, 2011; Sridhar et al., 2013) and to harvest hPSCs for subsequent differentiation. It focuses on the use of mTeSR1 medium and Matrigel to maintain hPSCs, although previous reports have demonstrated the ability to maintain hPSCs in alternate systems such as fibroblast feeder cells (Meyer et al., 2009, 2011; Sridhar et al., 2013). Cells are maintained on Matrigel-coated six-well culture plates and are split when confluency reaches ˜70%. This will aid in preventing spontaneous differentiation of cells due to overgrowth, while ensuring that an abundant amount of cells can be collected for directed differentiation. Typically, hPSCs are expanded at a ratio of 1:6, with a single well of cells capable of seeding an entire six-well plate. The starting population of hPSCs should display a tightly clustered and bright morphology and exhibit immunoreactivity to pluripotency markers.

### Materials

hPSCs growing on Matrigel™-coated six-well plate 2 mg/ml dispase solution (see recipe) DMEM/F12, 1:1 (Life Technologies)
mTeSR™1 (Stemcell Technologies)
Inverted light microscope 15-ml conical tubes
Matrigel™-coated six-well plate (see recipe)

### Passage Undifferentiated Cells

1) Place a six-well plate of hPSCs under an inverted light microscope and mark areas of spontaneous differentiation.
2) Transfer plate to a biological safety cabinet and use a 1-ml pipet tip to scrape away any cells from the marked areas of differentiation.
3) Aspirate medium from wells and replace with 1 ml/well of 2 mg/ml dispase solution. Transfer to incubator for 10 min and monitor every few minutes to ensure cells are beginning to detach from the culture surface. If dispase is sufficiently warmed to 37° C., this process should not take more than 15 min.
4) Once a majority of cell clusters display curled edges, immediately remove dispase by aspiration.
5) Wash cells once with 1 ml DMEM/F12 per well, adding the medium to the side of the well, not directly onto the cells, so that colonies are not prematurely detached from the surface.
6) Aspirate DMEM/F12 and add another 1 ml DMEM/F12 per well, this time with force directly onto the cells to detach the colonies. Repeat several times, if needed. It is better to forcefully dislodge colonies by pipetting three or four times than to gently agitate any more than this. Minimizing the amount the cells are broken up is key to ensuring maximum survival.
7) Transfer cells from wells that will be expanded to one 15-ml conical tube and cells from wells for retinal differentiation to another 15-ml conical tube. Proceed to step 8 for expansion or to Basic Protocol 2 for differentiation.
   Expand Undifferentiated hPSCs
8) Allow cells to settle to the bottom of the tube by gravity or by centrifuging 1 min at 100×g. Aspirate supernatant, taking care not to disturb the cell pellet.
9) Resuspend cells in mTeSR™1 medium so that each new well will receive 500 μl suspension (i.e., to passage one well of hPSCs to six new wells, use 3 ml mTeSR1). Break up clusters by pipetting forcefully four to five times with a 5 ml serological pipet to give clusters that are ˜100 to 150 μm in diameter. Note that cell lines can vary in ease of breaking up cell clusters. If clusters are not broken up sufficiently, increase pipetting during the next passage. It is better that clusters remain large than to be broken up too much.
10) Pipet 500 μl undifferentiated cell suspension at a 90° angle into each well of a freshly prepared Matrigel™-coated six-well plate.
11) Transfer to the incubator and agitate plates in side-to-side followed by front-to-back motions to ensure even distribution of cells. Be sure to pause briefly between series of agitations to ensure that cells are evenly dispersed across the well rather than accumulated in the middle.

Change medium daily (2 ml/well) until the next passage, typically within 4 to 5 days.

Basic Protocol 2: Induction of hPSCs to a Primitive Anterior Neuroepithelial Fate

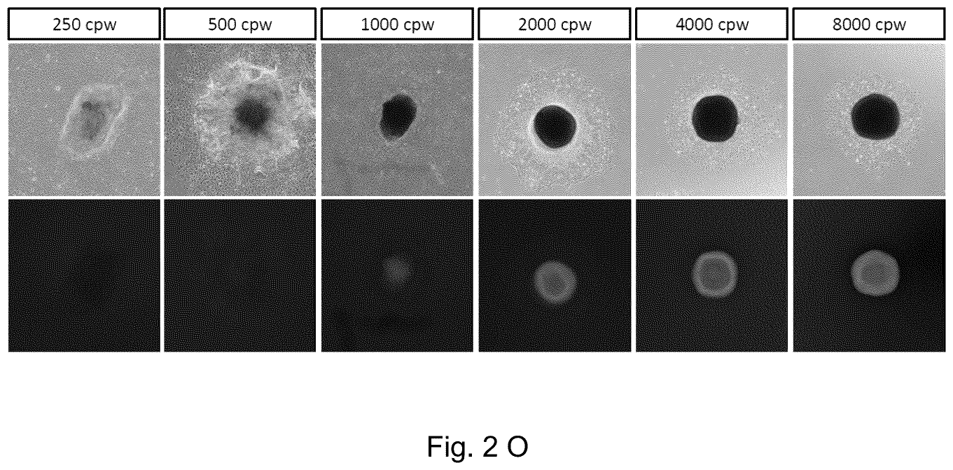

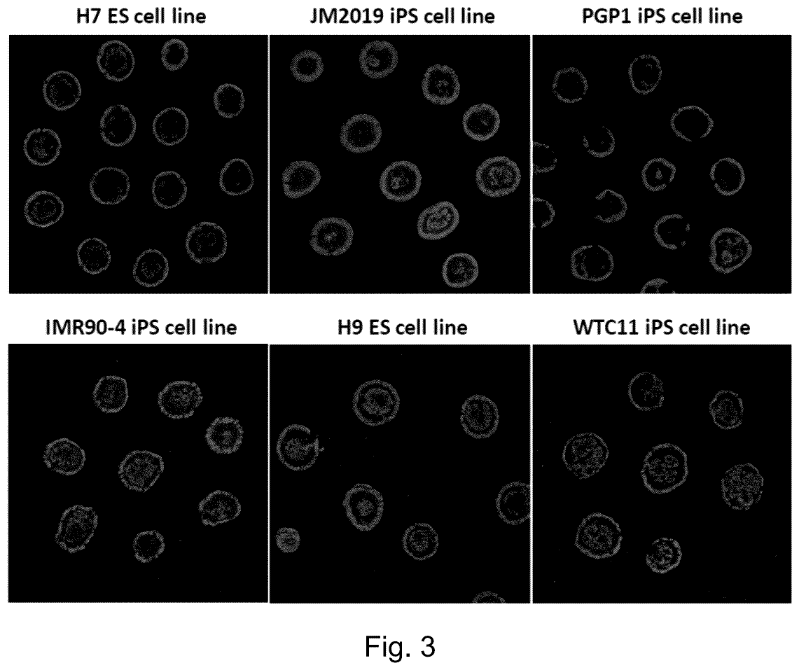

As retinal cells are derived from a pluripotent source through a stepwise process in vivo (Oliver and Gruss, 1997; Livesey and Cepko, 2001; Marquardt and Gruss, 2002; Zhang et al., 2002), hPSCs should be differentiated through analogous stages of differentiation, including a primitive anterior neural fate, an optic vesicle stage, and eventually a retinal and/or RPE fate (Meyer et al., 2009, 2011; Sridhar et al., 2013; Zhong et al., 2014). To initiate this stepwise process, embryoid bodies (EBs) are kept in suspension to begin differentiation for the first 7 days. This phase requires a slow transition out of mTeSR™1 medium into NIM and maintenance in a T75 flask. After 7 total days of differentiation, EBs are plated onto six-well culture plates to allow for further neural differentiation. This can be accomplished by addition of 10% FBS for the first 24 hr of plating to ensure that cells adhere to the wells. EBs are maintained in NIM until day 16. By day 10, they can be characterized by a larger, more uniform appearance as well as the expression of typical neural and eye-field transcription factors (FIG. 3).

### Materials

Harvested and washed undifferentiated hPSCs (see Basic Protocol 1, steps 1-8) mTeSR1 (Stemcell Technologies)
Neural induction medium (NIM; see recipe)
Fetal bovine serum (FBS)
T75 culture flask (Falcon)
15-ml conical tube
Six-well culture plate

### Generate Embryoid Bodies

1. Day 0: Allow cells to settle to the bottom of the tube by gravity. Aspirate supernatant and gently resuspend pellet in a 3:1 mixture of mTeSR™1/NIM. Transfer to a T75 flask and place in the incubator overnight. Day 0 is defined as the day cells are lifted from the Matrigel™-coated plate.
2. Day 1: Transition cells to a 1:1 mixture of mTeSR™1/NIM as described in step 1.
3. Day 2: Transition cells to a 1:3 mixture of mTeSR™1/NIM as described in step 1.
4. Day 3: Transition cells to complete NIM as described in step 1. Continue culturing until day 7, replacing medium with fresh NIM every other day.
5. Day 7: Collect EBs in a 15-ml conical tube and allow them to settle by gravity, typically within 5 min.
6. Add 2 ml NIM to each well of an uncoated six-well plate. In general, EBs derived from five wells of undifferentiated cells will plate on one six-well plate, with ^{˜}30 to 40 EBs per well.
7. Aspirate supernatant from the EBs, being careful not to disturb the cell pellet. Resuspend EBs in NIM so that each well will receive 200 μl suspension (e.g., 1.2 ml NIM for one plate), and add cells to the six-well plate accordingly.
8. Add 250 μl of FBS to each well (final concentration, ˜10%) to promote cell attachment.
9. Place in the incubator overnight, agitating in a side-to-side and then front-to-back manner to ensure an even distribution of cells within each well.
10. Day 8: Aspirate medium and add 2 ml fresh NIM without FBS to each well.
11. Return to incubator and change medium every other day until day 16 of differentiation. At this point, cells will have acquired a primitive anterior neuroepithelial fate.

### Basic Protocol 3: Differentiation of Primitive Anterior Neuroepithelial Cells to a Retinal Pigment Epithelial Fate

During normal development, the RPE is the first retinal cell type to be specified from a more primitive source. The RPE layer develops in a manner that is distinctly separate from the neural retinal populations of cells, and is known to be specified in the absence of factors instrumental in directing a neural retinal fate (Fuhrmann et al., 2000; Shibahara et al., 2000; Martinez-Morales et al., 2003). Likewise, RPE cells generated from hPSCs are found to differentiate through a similar process in which RPE cells are often found in close proximity to, although distinctly separate from, neural retinal populations (Capowski et al., 2014; Zhong et al., 2014). hPSC-derived primitive anterior neuroepithelial cells on six-well plates can be used to generate a highly purified population of RPE. These hPSC-derived RPE cells can be readily identified by their accumulation of pigmentation and their distinct hexagonal morphology (FIG. 4), and have been successfully generated by many groups in recent years (Vugler et al., 2008; Buchholz et al., 2009, 2013; Carr et al., 2009; Meyer et al., 2009, 2011; Liao et al., 2010; Maruotti et al., 2013; Rowland et al., 2013; Singh et al., 2013a; Sridhar et al., 2013; Capowski et al., 2014; Ferrer et al., 2014).

### Materials

Primitive anterior neuroepithelial cells in six-well plate (see Basic Protocol 2)
Retinal differentiation medium (RDM; see recipe)
Epidermal growth factor (EGF)
Fibroblast growth factor 2 (FGF2)
Heparin
Inverted light microscope
Tungsten needle, pipet tip, or other pointed object
Laminin/polyornithine-coated coverslips in a 4- or 24-well plate (see Support Protocol)
1) Day 16 of differentiation: Change medium from NIM to RDM and return to incubator. Continue culturing and changing medium every 2 to 3 days until distinct populations of RPE cells are readily observed and can be isolated (typically within 60 total days of differentiation).
2) Day of RPE isolation: Using a microscope, identify a suitably pure area of RPE based on pigmentation and hexagonal “cobblestone-like” morphology.
3) Using a pointed object (e.g., tungsten needle, pipet tip), gently scratch away an area around the region of cells to be microdissected, freeing the RPE cells.
4) Using a P100 pipettor, transfer the freed cluster of RPE cells in 50 μl RDM to a laminin/polyornithine-coated coverslip in a 4- or 24-well plate. Typically, one cluster of RPE cells should be sufficient for one coverslip.
5) Repeat this process for as many coverslips as needed.
6) Transfer the plate to the incubator and allow RPE to attach overnight. After overnight incubation, RPE clusters should have adhered to the coverslips.
7) Add 500 μl RDM supplemented with 20 ng/ml EGF, 20 ng/ml FGF2, and 2 μg/ml heparin to allow for proliferation of RPE cells. Continue culturing and replacing medium and growth factors every 2 days. RPE cells are expected to double in number approximately every 36 hr. Within 7 to 10 days, RPE cells will have lost most of their pigmentation and hexagonal shape, and will have occupied most of the coverslip.
8) To allow for maturation and reacquisition of RPE morphology, replace RDM containing growth factors with RDM alone and maintain for 2 to 3 weeks, or until a desired stage of RPE maturation is reached. RPE differentiated in this fashion may be maintained for at least two weeks.

### Cells

### Basic Protocol 4: Differentiation and Long-Term Maintenance of Retinal Progenitor

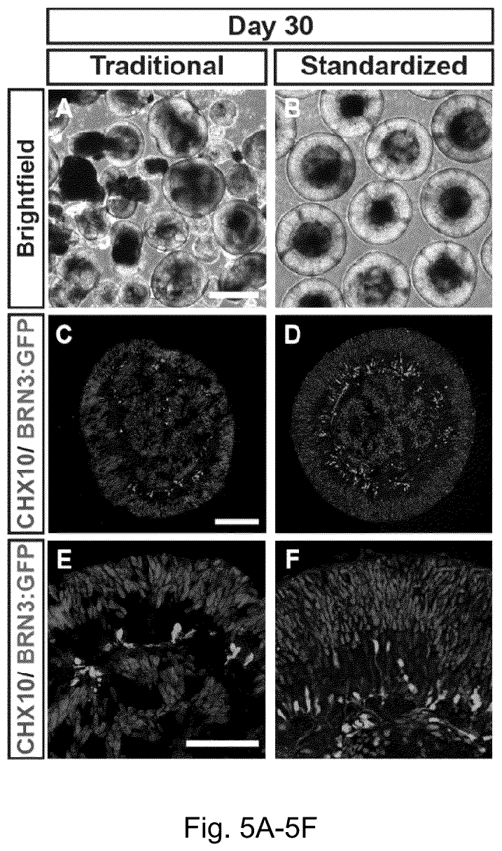

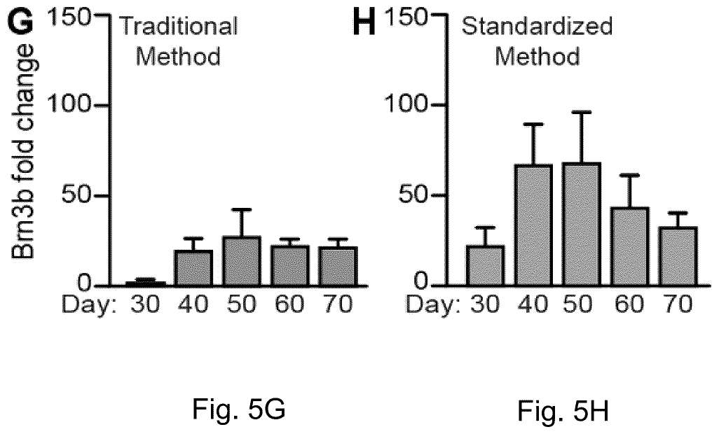

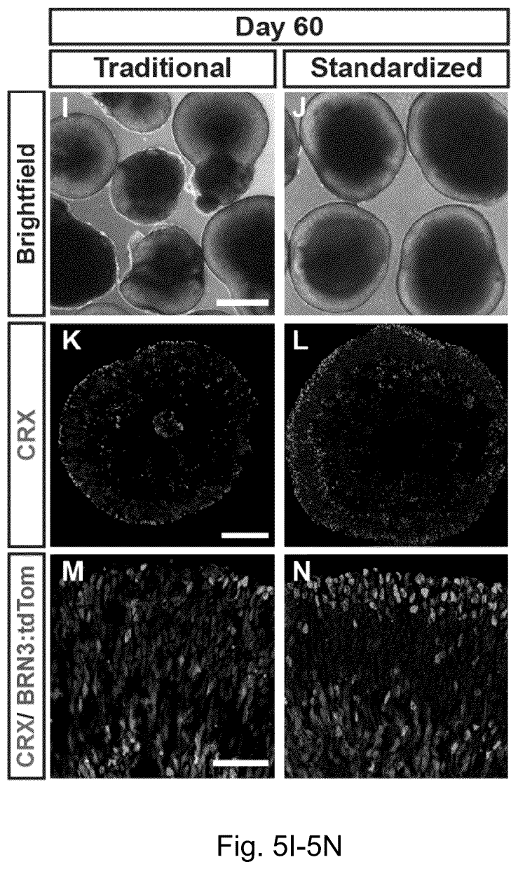

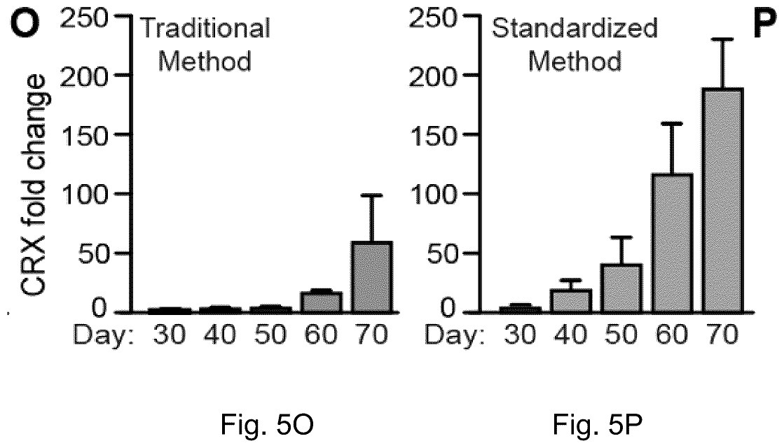

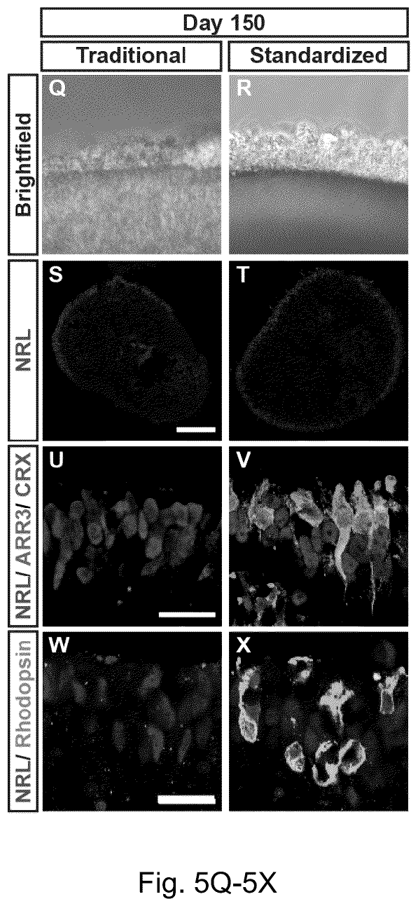

During in vivo development, after cells have adopted a primitive anterior neural phenotype, a subset of cells are known to acquire a retinal fate beginning with the optical vesicle stage of retinogenesis, and are characterized by numerous retinal-associated features that distinguish these cells from other neural lineages (Belecky-Adams et al., 1997; Rowan et al., 2004; Horsford et al., 2005; Bharti et al., 2008). Once this optic vesicle identity has been established, all mature retinal cell types (cones, rods, retinal ganglion cells, and so on) will eventually arise. Likewise, hPSCs can progress through an optic vesicle-like intermediary (FIG. 5), eventually yielding all of the major cell types of the retina (Meyer et al., 2009, 2011; Sridhar et al., 2013; Capowski et al., 2014; Phillips et al., 2014). To accomplish this, cells are lifted from the culture surface at 16 days of differentiation and maintained in floating suspension in RDM to allow for development of a three-dimensional optic vesicle-like structure. Retinal and non-retinal cells can then be manually separated and maintained until the desired stage of differentiation is reached.

### Materials

Primitive anterior neuroepithelial cells in six-well plate (see Basic Protocol 2)
Retinal differentiation medium (RDM; see recipe)
60×15 mm polystyrene Petri dishes
Six-well plates (Falcon)
   Generate Neurospheres from Primitive Anterior Neuroepithelial Cells
1) Day 16 of differentiation: Using a P1000 pipettor, draw up 1 ml medium from a well of cells and dislodge the center of each aggregate from the plate by vigorously pipetting the medium directly at the center of the aggregate. Repeat several times, if needed, to dislodge the center and leave a ring of peripheral cells possessing a flattened appearance. It is better to pipet forcefully five to six times instead of gently pipetting any more than this, as excessive pipetting results in cell death and reduced yield of neurospheres.
2) Transfer dislodged aggregates to a 15-ml conical tube and allow to settle by gravity or by centrifugation for 1 min at 100×g.
3) Aspirate supernatant and resuspend cells in 5 ml RDM.
4) Transfer cell suspension to a 60-mm dish and return to the incubator. Continue culturing for up to 25 total days of differentiation, changing medium every 2 to 3 days. By 20 to 25 total days of differentiation, two populations of neurospheres will begin to emerge: retinal neurospheres that have a golden ring around the outside, and non-retinal forebrain neurospheres that have a darker appearance and lack this golden ring. These morphological differences can easily be observed with an inverted microscope under a 4× objective. By 30 total days of differentiation, distinct transcriptional profiles emerge that distinguish these two populations (FIG. 5). Enrichment of retinal neurospheres should be performed by day 25 of differentiation.

### Manually Enrich Retinal Neurospheres

5) While viewing the cells under an inverted light microscope with a 4× objective, swirl the plate gently in a circular motion to collect cells in the middle of the dish.
6) Looking at the neurospheres through the microscope, gently gather retinal neurospheres based on their bright outer ring appearance using a P20 pipettor and transfer them to a 15 ml conical tube containing 5 ml RDM. Repeat until all retinal neurospheres have been collected in the same tube.
7) Transfer retinal neurospheres (along with the medium) to a 60-mm dish and return to the incubator. Non-retinal neurospheres may be similarly maintained for neuronal cultures, if desired, or discarded at this stage.

### Maintain Retinal Neurospheres

8) Culture retinal neurospheres until the desired stage of retinal differentiation is reached, changing the medium every other day as follows:
a. Tilt the plate towards you to allow the medium and neurospheres to collect in the bottom half of the plate.
b. While maintaining the tilt of the plate, use a P1000 pipettor to transfer 1 ml medium containing as many neurospheres as possible to a 15 ml conical tube.
c. Collect the remaining medium and rinse over the entire surface of the plate to collect any remaining neurospheres. Transfer this remaining medium to the 15-ml conical tube.
d. Allow neurospheres to settle to the bottom of the tube, then aspirate the supernatant.
e. Resuspend neurospheres in 5 ml fresh RDM and transfer back to the dish. Retinal ganglion cells are expected to arise after 40 total days of differentiation, and photoreceptor progenitor cells are expected to arise within 70 total days of differentiation.
   c. Protocol 5: Induction of Retinal Progenitors to Specific Retinal Subtypes

Previous studies have demonstrated that hPSC-derived retinal progenitor cells have the ability to yield all major classes of retinal cells, including photoreceptors (Lamba et al., 2006, 2010; Osakada et al., 2008; Meyer et al., 2009, 2011; Mellough et al., 2012; Gonzalez-Cordero et al., 2013; Tucker et al., 2013a, 2013b; Reichman et al., 2014; Zhong et al., 2014) and retinal ganglion cells (Lamba et al., 2010; Meyer et al., 2011; Sridhar et al., 2013; Zhong et al., 2014). In order to derive these various cell types, retinal neurospheres must be maintained in differentiating cultures for extended periods of time. Within 90 days of total differentiation, neural retinal cell types including photoreceptors and retinal ganglion cells can be identified. In order to analyze cells by immunocytochemistry, neurospheres should be dissociated with ACCUTASE (a cell detachment solution of proteolytic & collagenolytic enzymes) and then plated onto laminin/polyornithine-coated coverslips. At this point, cells demonstrate the presence of a wide variety of retinal-specific transcription factors and distinct neuroretinal morphologies, such as neurite outgrowth and/or axonal and dendritic arborization typical of retinal ganglion cell or photoreceptor morphologies.

### Materials

Retinal neurospheres at 40 days total differentiation (see Basic Protocol 4)
ACCUTASE (a cell detachment solution of proteolytic & collagenolytic enzymes; BD Biosciences)
Retinal differentiation medium (RDM; see recipe)
Laminin/polyornithine-coated coverslips (see Support Protocol)
Inverted light microscope
1.5 ml tube
1) Place the plate of retinal neurospheres in suspension under a microscope and swirl in a circular motion to gather cells towards the center of the field of view.
2) Gather neurospheres that will be used for dissociation and plating and transfer to a 1.5 ml tube. Two to three neurospheres per coverslip typically provide enough density for sufficient plating and microscopy.
3) Allow neurospheres to settle to the bottom of the tube and gently remove excess medium with a pipet.
4) Add 200 μl ACCUTASE (a cell detachment solution of proteolytic & collagenoloytic enzymes) and transfer to a 37° C. water bath.
5) Every 10 min, remove tube from water bath and forcefully agitate cells about four or five times with a P100 pipettor (on 50 μl setting) to break up the cells.
6) Repeat step 5, if needed, until aggregates are the desired size (ideally, ˜200 to 400 μm in diameter). Viability is greatly increased if cells are dissociated to yield small aggregates of cells rather than a single-cell suspension.
7) Centrifuge suspension for 1 min at 100×g. Gently remove supernatant with a pipet.
8) Resuspend cells in enough RDM to ensure that each coverslip receives 50 μl suspension.
9) Pipet 50 μl suspension onto each laminin/polyornithine-coated coverslip.
10) Transfer to incubator and allow cells to adhere overnight.
11) If cells are to be maintained on coverslips for further differentiation, add 500 μl RDM the following day and every other day thereafter until the desired stage of differentiation is reached. If cells are to be fixed immediately for immunocytochemistry, no additional medium should be added.
   Support Protocol: Coverslips with Laminin and Poly-D-Ornithine

This brief protocol explains how to coat coverslips for use in Basic Protocols 3 and 5. Poly-D-ornithine increases adhesion of cells to the coverslip, and laminin promotes cell growth. Cells grown on laminin/polyornithine-coated coverslips can easily be utilized for immunocytochemical analysis and readily transferred to slides for visualization by microscopy.

### Materials

12-mm glass coverslips, washed with ethanol and subsequently autoclaved 4- and/or 24-well plates
100 μg/ml poly-D-ornithine solution (see recipe)
20 μg/ml laminin solution (see recipe)
1) Transfer one coverslip to each well of a 4- or 24-well plate and ensure that it lies flat on the bottom of the well.
2) Pipet 100 μl poly-D-ornithine onto the center of each coverslip. Let sit at room temperature for 30 min, ensuring the poly-D-ornithine remains on the coverslip.
3) Remove solution and wash each well with 1 ml sterile water. Repeat two more times.
4) Remove the third wash and check coverslips under a microscope to make sure any precipitate has been washed away. Continue rinsing if any residue remains.
5) Remove last wash and allow coverslips to air dry in a biological safety cabinet overnight. Be sure to leave the hood fan on and the sash slightly open.
6) Remove dry coverslips from hood. If desired, polyornithine-coated coverslips can be stored at room temperature for at least 1 month.
7) Add 50 μl of 20 μg/ml laminin directly to the center of each poly-D-ornithine-coated coverslip.
8) Transfer plates to the incubator and let stand at least 4 hr or overnight to allow thorough coating of coverslips.
9) Aspirate excess laminin just before addition of cell suspension. Laminin is not expected to have a long half-life, and thus laminin-coated slides should be used shortly after preparation.

### Reagents and Solutions

All solutions should be made in a biological safety cabinet and filtered through a Steriflip™ or bottle top filter to ensure solutions are sterile

Dispase Solution, 2 mg/ml: Dissolve 2 mg/ml dispase powder (Life Technologies) completely in DMEM/F12 (1:1, Life Technologies). Warm for at least 20 min in a 37° C. water bath, then filter sterilize. Store up to 2 weeks at 4° C.

Laminin Solution, 20 μg/ml: Starting with a 1 mg/ml stock, dilute laminin 1:50 in cold DMEM to a final concentration of 20 μg/ml. Store up to 1 month at 4° C.

Matrigel-coated Plates: Dilute Matrigel (hESC-qualified, BD Biosciences) according to manufacturer's specifications in DMEM. Coat six-well culture plates (e.g., Falcon) by adding 1 ml Matrigel per well and placing in a 37° C., 5% CO2 incubator for at least 1 hr. Aspirate excess Matrigel from plates, then add 2 ml mTeSR1 medium (Stemcell Technologies) to each well. Keep at 37° C. and use within 8 hr.

### Neural Induction Medium (NIM)

489.5 ml DMEM/F12 (1:1, Life Technologies)
5 ml N2 supplement (Life Technologies)
5 ml MEM non-essential amino acids
0.5 ml 2 mg/ml heparin
Filter sterilize
Store up to 1 month at 4° C.
   Poly-D-Ornithine Solution, 100 μg/ml

Tap a 10-mg bottle of poly-D-ornithine (Sigma) on the surface of the hood and open carefully as to not lose any powder. Slowly pipet 1 ml sterile water into the bottle, replace the cap, and shake vigorously. Carefully remove cap and transfer solution to an autoclaved 250-ml beaker. Repeat this process about four more times to remove all traces of poly-D-ornithine from the bottle. Then, add sterile water to the beaker to bring the volume to 100 ml. Pipet up and down to mix thoroughly. Transfer aliquots to 50-ml conical tubes. Filter sterilize each aliquot using a Steriflip™ 0.2-μm filtering device. Store up to 6 months at 4° C.

### Retinal Differentiation Medium (RDM)

240 ml DMEM/F12 (1:1, Life Technologies)
240 ml DMEM (Life Technologies)
10 ml B27 supplement without vitamin A (Life Technologies)
5 ml MEM non-essential amino acids
5 ml antibiotics
Filter sterilize
Store up to 1 month at 4° C.

The invention will be more fully understood upon consideration of the following non-limiting Examples. It is specifically contemplated that the methods disclosed are suited for pluripotent stem cells generally.

### EXAMPLES

### Example 1-Method of Obtaining Three-Dimensional Human Retinal Organoid Tissue Yielding More Uniform Organoids at Higher Efficiency

### Material & Reagents:

Rock inhibitor-Y-27632 Stemgent-040012-02
96 U low adhesion bottom plates-Thermo Fisher-_174925

### Medium

### NIM (Neural Induction Medium)

484.5 mL DMEM/F12 (Life Technologies, Catalog #11330-057)
5 mL N2 Supplement (Life Technologies, Catalog #17502048)
5 mL MEM NEAA (Life Technologies, Catalog #11140050)
5 mL penicillin-streptomycin (Life Technologies, Catalog #15240062)
0.5 mL Heparin-2 mg/mL (Sigma-Aldrich, Catalog #H3149-50KU)
Combine reagents and filter. Store up to 1 month at 4° C.

### RDM (Retinal Differentiation Medium)

240 mL DMEM/F12 (Life Technologies, Catalog #11330-057)
240 mL DMEM (Life Technologies, Catalog #12430062)
10 mL B27 Supplement (Life Technologies, Catalog #17504001)
5 mL MEM NEAA (Life Technologies, Catalog #11140050)
5 mL penicillin-streptomycin (Life Technologies, Catalog #15240062)
Combine reagents and filter. Store up to 1 month at 4° C.

### RMM (Retinal Maturation Medium)

215 mL DMEM/F12 (Life Technologies, Catalog #11330-057)
215 mL DMEM (Life Technologies, Catalog #12430062)
50 mL Fetal Bovine Serum (FBS)
10 mL B27 Supplement (Life Technologies, Catalog #17504001)
5 mL MEM NEAA (Life Technologies, Catalog #11140050)
5 mL penicillin-streptomycin (Life Technologies, Catalog #15240062)
1× Glutamax (Life Technologies, Catalog #35050061)
Combine reagents and filter
Store up to 1 month at 4 degrees

### Add Fresh Each Media Change:

100 μM Taurine fresh each media change (Sigma-Aldrich, Catalog #T0625)

### ARMM (Advanced Retinal Maturation Medium)

215 mL DMEM/F12 (Life Technologies, Catalog #11330-057)
215 mL DMEM (Life Technologies, Catalog #12430062)
50 mL Fetal Bovine Serum (FBS)
10 mL B27 Supplement (Life Technologies, Catalog #17504001)
5 mL MEM NEAA (Life Technologies, Catalog #11140050)
5 mL penicillin-streptomycin (Life Technologies, Catalog #15240062)
1× Glutamax (Life Technologies, Catalog #35050061)
Combine reagents and filter
Store up to 1 month at 4 degrees

### Add Fresh Each Media Change:

100 μM Taurine fresh each media change (Sigma-Aldrich, Catalog #T0625)
1 μM Retinoic Acid
DHA in 0.1% FA free BSA (50 μM)—at 4:1 carrier ratio
BDNF (20 ng/mL) [brain derived neurotrophic factor]

### Differentiation Day-1: Aggregation

### Preparation:

Cultured hPSCs are 50%-80% confluent.
Refeed cells 1-2 hours before differentiation culture if culture medium is orange or yellow-refeed in the morning before starting.
Obtain mTeSR1 medium, an aliquot of accutase, and cloner, to room temperature.
Prepare mTeSR medium with 20 μM Y-27632 by adding 22 μL of Y-27632 to 11 mL of mTeSR medium in a 15 mL conical tube for 1×96 U bottom plate (stock 10 mM-Y-27632).
Prepare two tubes of mTeSR medium-1 mL mTeSR medium in each of the two 1.5 mL micro centrifuge tubes.
Invert all tubes several times to mix well.

### Method and Steps:

1. Aspirate the spent mTeSR medium from one well of human pluripotent stem cells (hPSCs) backplate and wash three times with DPBS/DMEM at RT.
2. Using a P1000 pipette, add 500 μL of accutase/accumax and incubate at 37 C for 5 min. While you wait, set up 500 μl tube for Trypan blue-add 30 μl of TB (use later at step 16).
3. Check for cell dissociation under a microscope. Note: DO NOT ASPIRATE ACCUTASE as cells will be floating.
4. Gently pipet 1 mL of mTeSR across the well to wash the cells off the plate (use circular rainbow motion)-pipette up and down 2-3 times.
5. Collect cells into a 2 mL round-bottom microcentrifuge tube and gently pipet up and down several times (8-10 times) to dissociate cells into single cells-(1.5 ml-500 μl accutase and 1 ml mTeSR).
6. Centrifuge tube with 1.5 ml balance at 200×g for 3 min.
7. Completely aspirate supernatant while keeping cell pellet undisturbed.
8. Resuspend the cell pellet with 1 mL of mTeSR. Gently pipet up and down (8-10 times) to mix cells and to ensure single cell dissociation.
9. Using the 500 uL microcentrifuge tube, mix 30 μL of cells with the 30 μL of trypan blue and determine the concentration of cells using the trypan blue-cell mixture via an automated cell counter.
10. Place coverslip on cell counter and add 9-10 μl to each side of the cell counter.
11. Count cells using automated cell counter, take an average of each side (live cell count only) and calculate the concentration of cells.
12. Add cell suspension into a reservoir and slowly add 100 μL of cell suspension into each well of a low-cell-adhesion 96-well V or U bottom plate using a multichannel pipet (p200).
13. Spin down the plates at 200×g for 6 min at RT and check morphology/cell number. Place plates in 37° C. incubator.

| Number of cells | Number of cells | Number of cells in 11 mLs - |
|---|---|---|
| per well | per mL | 1 × 96 U plate |
| 1,000 | 10,000 | 110 × 10³ |
| 2,000 | 20,000 | 220 × 10³ |
| 3,000 | 30,000 | 330 × 10³ |
| 4,000 | 40,000 | 440 × 10³ |
| 5,000 | 50,000 | 550 × 10³ |

For example, if the cell suspension contains 2×10⁶ cells/mL (2000×10³), dilute 165 μL of cell suspension (330×10³ divided by 2000×10³) in ˜11 mL of mTeSR medium. Gently invert tube several times to mix.

Differentiation Day 0: Addition of mTeSR Medium
1. Make up 1:1 media (NIM: mTeSR)
2. Add 100 μL of mTeSR:Neural induction media (1:1) to each well-3:1 ratio overall.
   Differentiation Day 1: Transfer to Differentiation Media to Drive Cells into Optic Lineage
1. Transfer aggregates/embryoid bodies to 60/100 mm dishes using open cut pipette tips (multi-channel)
2. Add mTeSR-1:1 ratio mTeSR:Neural induction media
   Differentiation Day 2: Transfer to Differentiation Media to Drive Cells into Optic Lineage
1. Transfer and wean off mTeSR-1:3 ratio mTeSR:Neural induction media
   Differentiation Day 3: Transfer to Differentiation Media to Drive Cells into Optic Lineage
1. Transfer to complete Neural induction media

### Differentiation Day 6: Addition of BMP4

1 Add Neural induction media+BMP4 (1.5 nM final concentration)

### Differentiation Day 8: Plate Cell Aggregates

1. Collect aggregates in a 15 mL conical tube and let them settle.
2. Add 500 μL of FBS to each well of a 6-well plate
3. Place 2 mLs of hPSC aggregates (with old NIM media from flask containing BMP4) into each well of the 6-well plate. Make sure that hPSC aggregates are evenly distributed.
4. Plate 10-20 aggregates per 6 well

### Differentiation Day 9: Half Media Change

1. Remove half (1 mL) of media from each well and replace with fresh NIM without FBS.

### Differentiation Day 12: Half Media Change

1. Remove half (1 mL) of media from each well and replace with fresh NIM without FBS.

### Differentiation Day 15: Full Media Change

1. Remove all media from each well and replace with 2 mL of fresh NIM without FBS.

### Differentiation Day 16: Media Transition to RDM Media

1. Using the tip of a P200 pipette tip and gently remove aggregates from the 6 well plate.
2. Transfer aggregates to a 15 mL conical tube and allow aggregates to settle at the bottom of the tube.
3. Aspirate the supernatant and wash with 2-3 mL of RDM supplemented with 1% FBS, aspirate again and resuspend in 5-10 mL RDM
4. Transfer the media with the aggregates to a 100 mm dish.

### Differentiation Day 18:

1. Supplement RDM with 1% FBS

### Differentiation Day 20:

1. Supplement RDM with 5% FBS

### Differentiation Day 20:

1. Supplement RDM with 5% FBS

### Differentiation Day 22:

1. Supplement RDM with 10% FBS+1× Glutamax

### Differentiation Day 25: Transition to Retinal Maturation Medium (RMM)

1. Supplement RDM with 10% FBS+1× Glutamax+Taurine (100 μM)
   after Differentiation Day 25 Change Media Every 2-3 Days.
Add Taurine fresh for each media change.

### Differentiation Day 50: Transition to Advanced Retinal Maturation Medium (ARMM)

1. Supplement RDM with 10% FBS+1× Glutamax+Taurine (100 μM), as well as 1 μM Retinoic Acid, 50 μM DHA in 0.1% FA free BSA) and 20 ng/mL BDNF
2. Change media every 2-3 days
   Differentiation Day 90: Remove Retinoic Acid from Advanced Retinal Maturation Medium (ARMM)
1. After 90 days of differentiation, do not add Retinoic Acid to ARMM media after 90 days of total differentiation
   Differentiation Day 90+-Advanced Retinal Maturation Medium (ARMM) (without Retinoic Acid)
1. Change media every 2-3 days

As organoid become advance in their differentiation and become larger in size, the media will turn yellow a lot faster. Changing media every 2 days is required at this point.

### Results

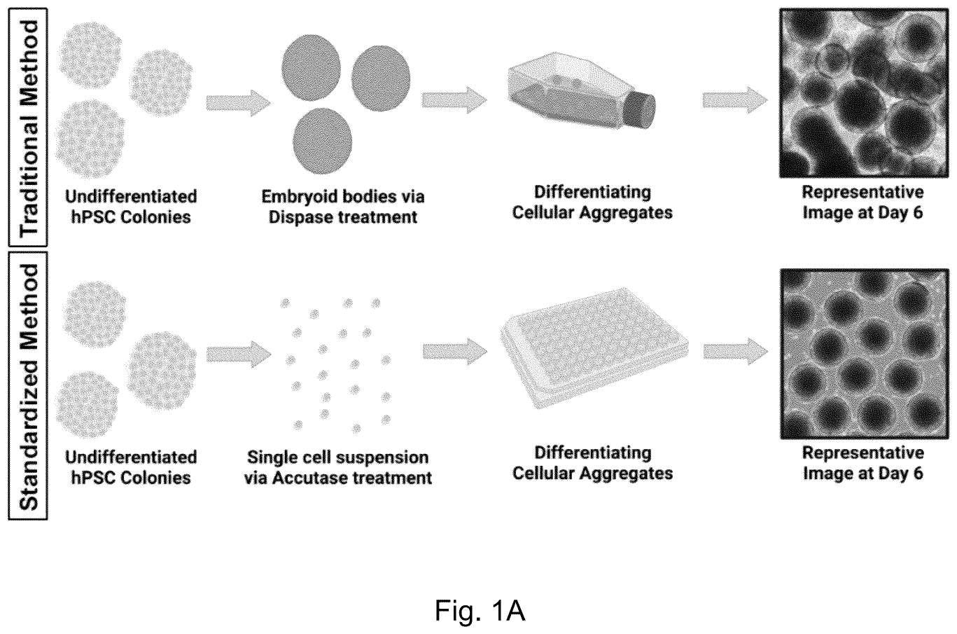

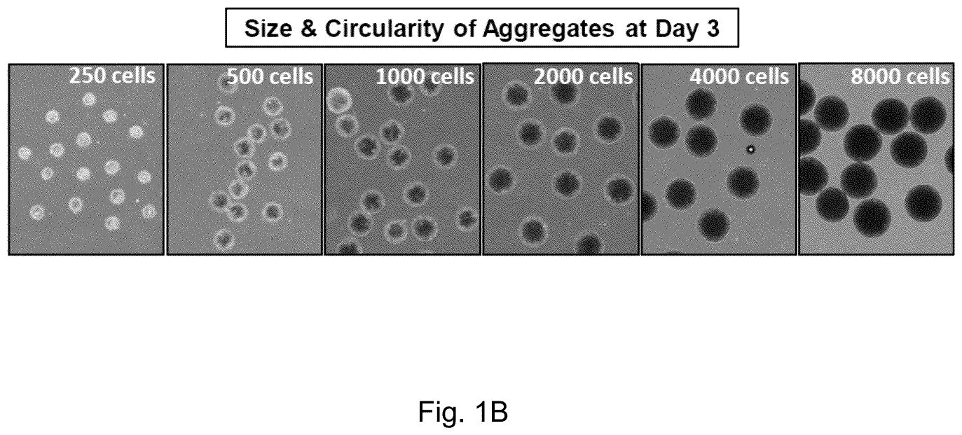

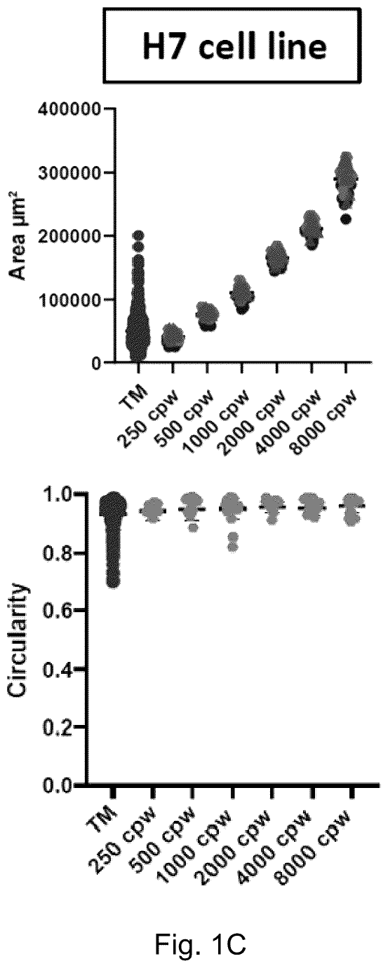

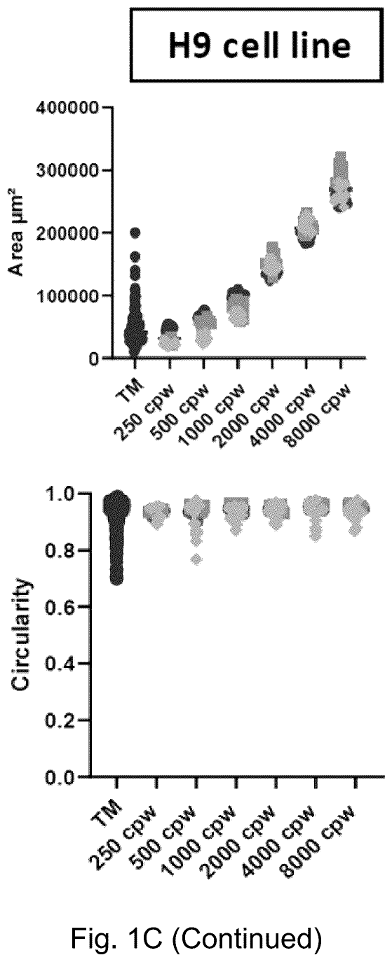

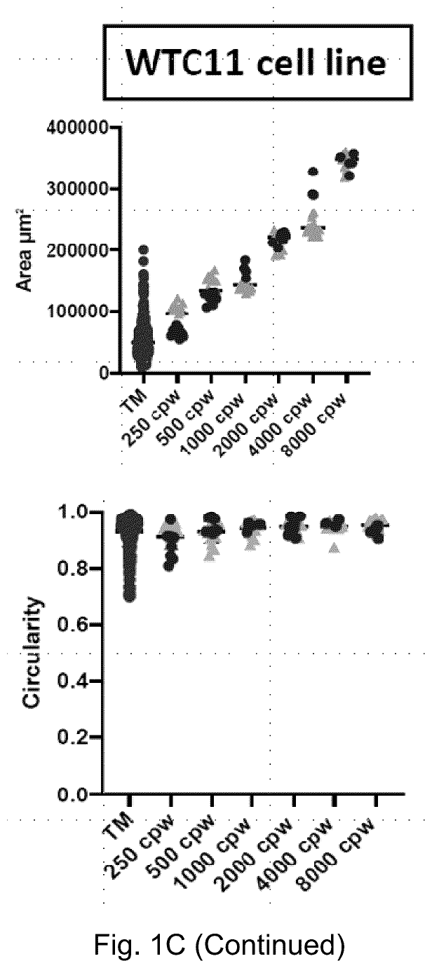

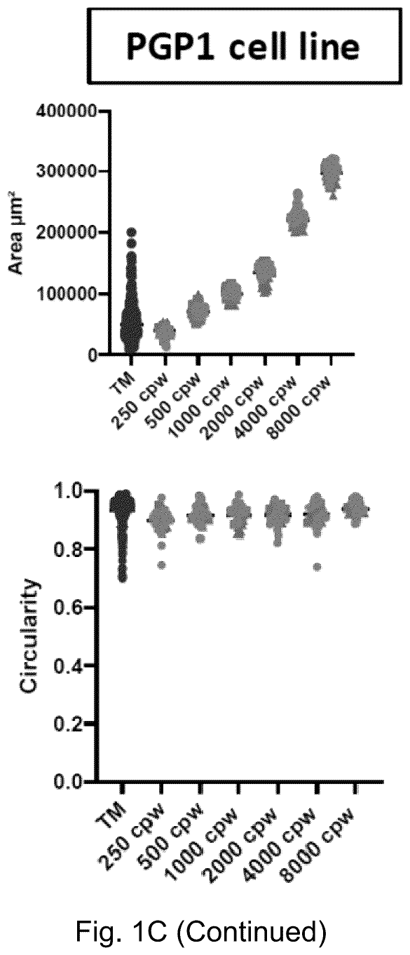

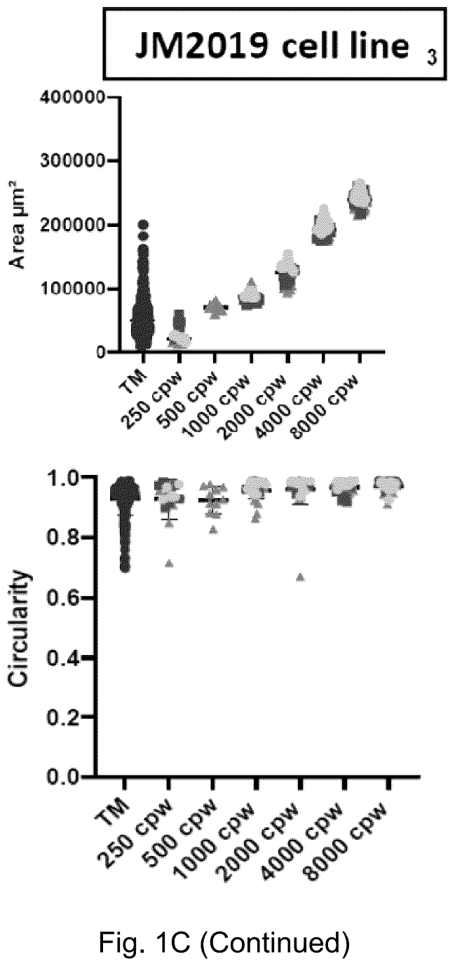

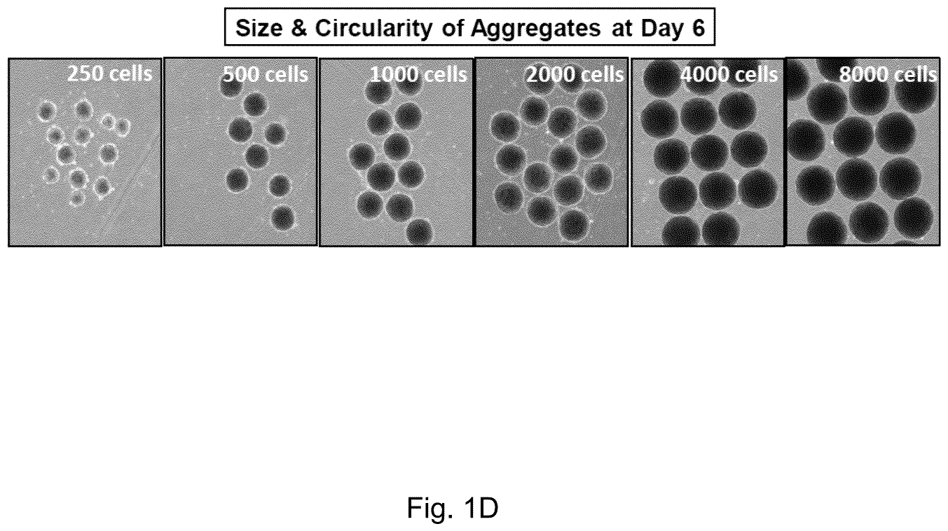

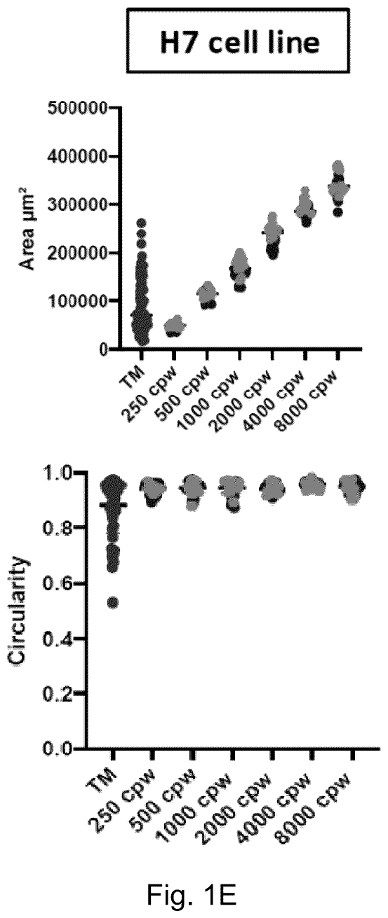

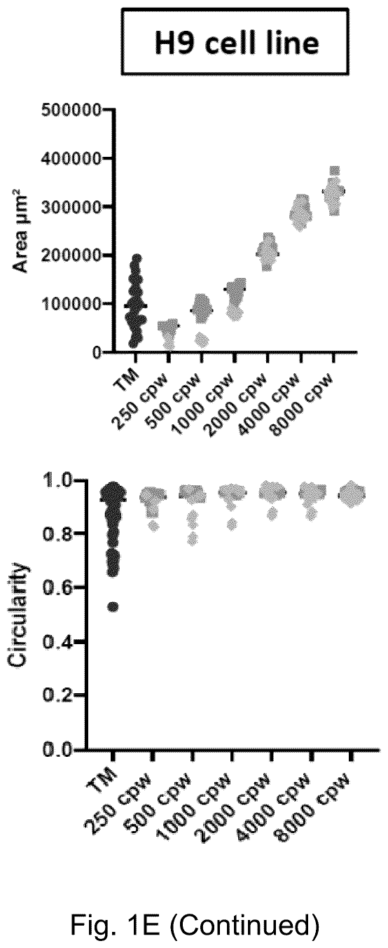

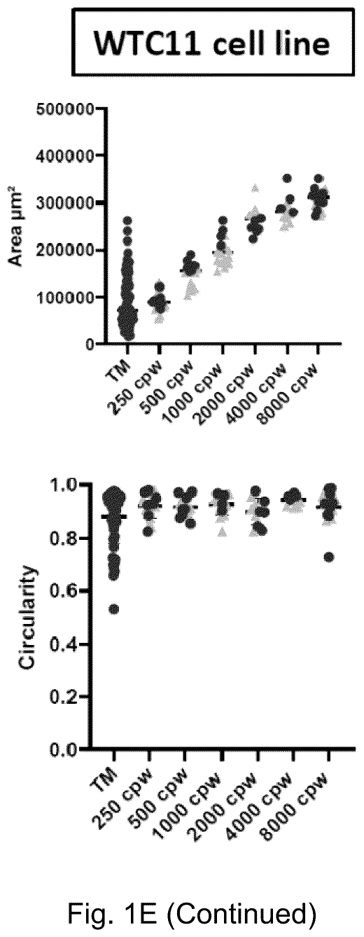

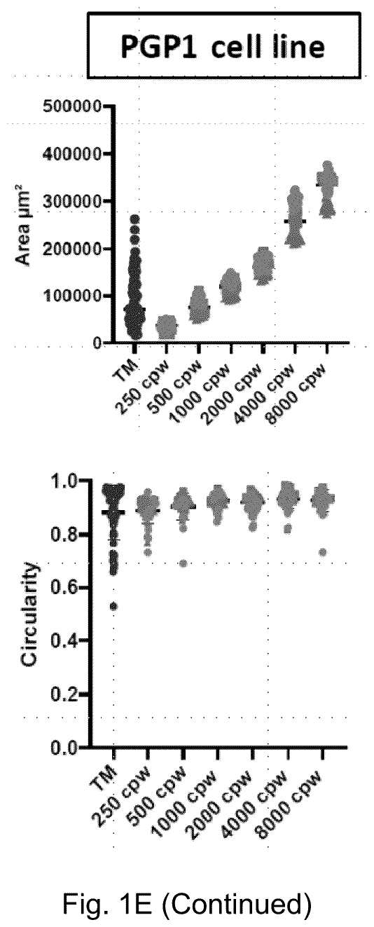

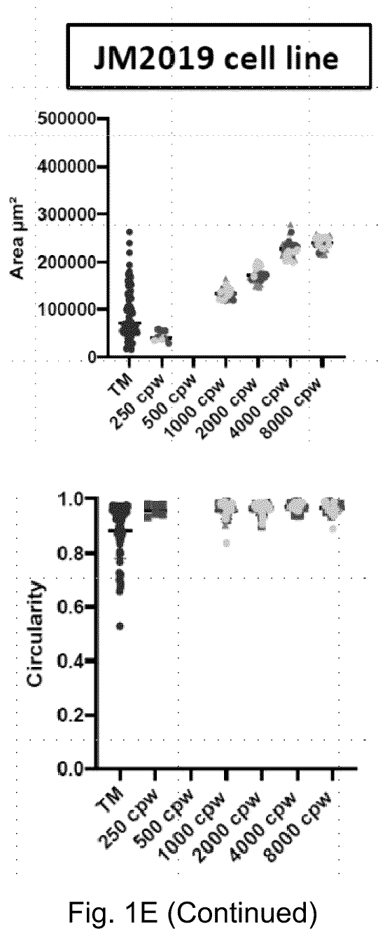

Traditional differentiation methods to dissociate pluripotent stem cells and form aggregates or organoid bodies, which often use a proteolytic enzyme like dispase (FIG. 1A) for dissociation, leads to cellular aggregates that are variable in both their size and shape. Our new, standardized dissociation methods for stem cells, particularly pluripotent stem cells, uses accutase, a proteolytic and collagenolytic enzyme, for differentiation. The novel standardized differentiation methods use accutase to dissociate the hPSCs into single cells and then allow control of both the size and shape of cellular aggregates (FIG. 1A). Using the standardized method, PSCs plated at densities ranging from 250 cells per well to 8000 cells per well resulted in greatly improved consistencies in size and circularity of the aggregates (FIGS. 1B-1E) compared to the traditional method of differentiation (FIGS. 1C and 1E). As seen in the figures both the size and circularity of the aggregates at the early stages of differentiation are more consistent (FIGS. 1B, 1D and 1F). In addition, non-PSC lines, such as ES and iPS cell lines, show that this standardized method is highly reproducible and consistent across multiple cell lines (FIGS. 1C and 1E).

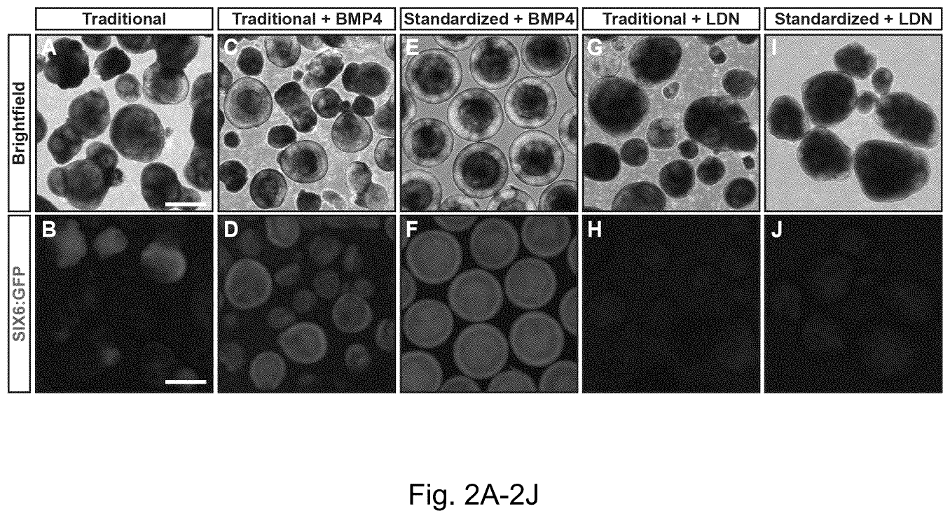

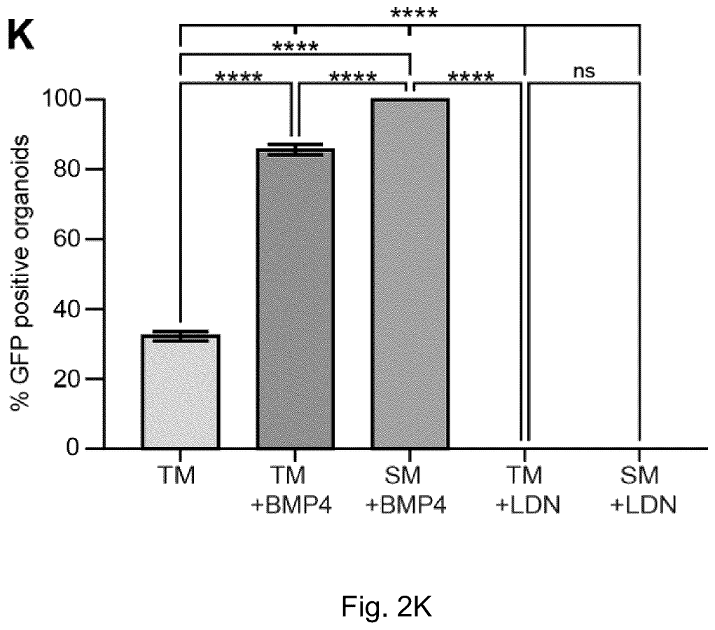

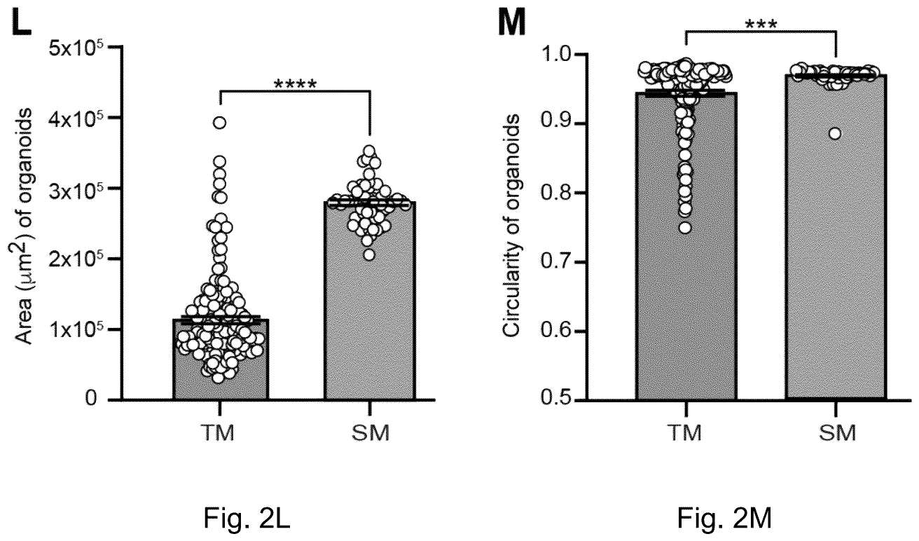

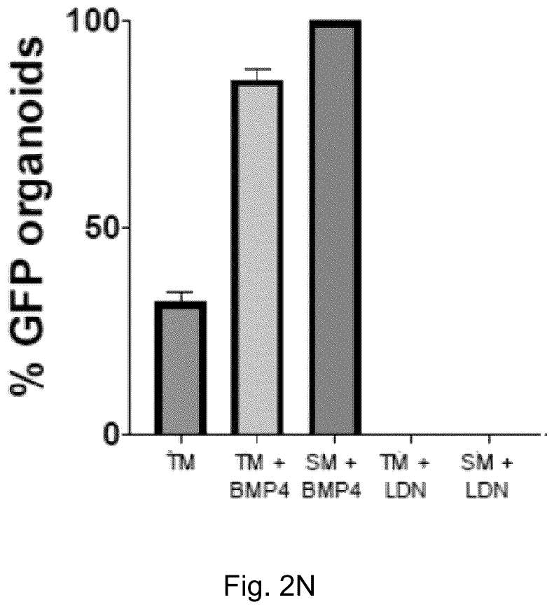

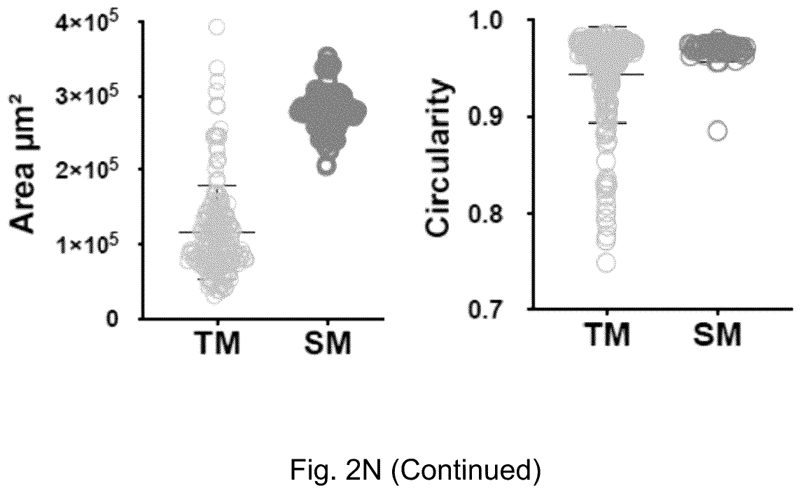

Biomarker analysis shows that compared to traditional differentiation methods, the standardized methods of differentiation provides more efficient differentiation (increase from 30% to 100%) and more consistent size and shape of aggregate or organoid development. SIX6, a specific retinal progenitor marker/transcription factor, was used for the quantification of the number of cellular aggregates that differentiate to retinal organoids between both the traditional and standardized methods of differentiation. Using the IMR90.4 cell line that has a SIX6-GFP reporter, hPSCs were differentiated using both the traditional and standardized methods until 25 Days of total differentiation. (FIGS. 2A-2J). Based on previous studies it was known that the precise timing of BMP4 treatment can enhance retinal organoid production. Accordingly, the generated aggregates were treated using both traditional and standardized differentiation protocols with BMP4 at Day 6. Untreated organoids, BMP4 treated organoids, and LDN treated organoids were compared by traditional method and standardized method. LDN is a small molecule BMP4 inhibitor, which allowed for confirmation of BMP4 expression. As can be seen in FIGS. 2A-2D, and 2K, approximately 30% of organoids express GFP using the traditional method and after treatment with BMP4 using the traditional method, around 80%-90% of the organoids express GFP. However, when using the standardized method of differentiation, after treatment with BMP4 (FIG. 2E), 100% of the organoids become retinal organoids, as shown by the GFP expression (FIGS. 2F and 2K). Conversely, when BMP4 signaling was blocked using LDN (FIGS. 2G-2J), organoids differentiated using both the traditional and standardized method do not express GFP (FIGS. 2H, 2J, 2K). Additionally, both the size and shape of the organoids were quantified at Day 25. The organoids differentiated using the standardized method of differentiation are more consistent in their size and circularity at Day 25 when compared to organoids differentiated using the traditional methods of differentiation (FIGS. 2L-2N). Overall, retinal organoids differentiated using the standardized method are not only more reproducible and consistent in both their size and shape at day 25, but that 100% of these organoids become retinal based on SIX6-GFP expression (FIG. 2L-2N). Moreover, cell seeding density may be a factor in the efficiency of retinal organoid formation. Cell seeding density of cells per well, at 250, 500, 1000, 2000, 4000, and 8000 cells per well, shows improved aggregate efficiency and quality of cell aggregate for differentiation. Aggregate differentiation is dependent upon the size of the initial cell aggregate generated, and at least 2000 cell seeds is optimal seeding to achieve 100% efficiency in retinal organoid differentiation with consistent size and shape of aggregate cells and organoids. (FIG. 20).

The standardized differentiation method for retinal organoids is reproducible across multiple stem cell lines. Using six different ES and iPS cell lines, H7 ES, JM2019 iPS, PGP1 iPS, IMR90-4 iPS, H9 ES, and WTC11 iPS, organoids were differentiated and stained for the retinal progenitor marker Chx10. All sections stain positive for the retinal marker Chx10 (FIG. 3; shown in red). Overall, this novel method shows that is highly reproducible across multiple stem cell lines.

The transcriptome of early stages of differentiation show surprising early cell fate determination events after treatment with either BMP4 or LDN at Day 6. For this experiment bulk mRNA-sequencing was used. RNA from hPSC aggregates were collected at Day 6 before any treatments, and then treated with either BMP4 or LDN at Day 8. At Day 25 that the organoids expressed GFP (FIG. 4A). We then analyzed the mRNA-seq data comparing: (1) Day 8 BMP4 with Day 6 untreated (FIG. 4B); (2) Day 8 LDN with Day 6 untreated (FIG. 4C); and (3) Day 8 LDN with Day 8 LDN (FIG. 4D). Comparing Day 8 BMP4 treated cells with Day 6 untreated, after only 2 days treatment with BMP4 there is an upregulation in the expression of retinal specific genes including SIX6, RAX, LHX9 VSX2 (CHX10), and PAX6 (FIG. 4B). Conversely when comparing the Day 8 LDN treatment with Day 6 untreated, there is an upregulation of more cortical/forebrain related genes including FOXG1 and MAP2 (FIG. 4C). Additionally, when comparing the Day 8 BMP4 treatment with Day 8 LDN, there is an increase in retinal specific genes including VSX2 (CHX10), SIX6, LHX9 and RAX, and a decrease in more cortical/forebrain related genes including FOXG1 and MAP2.

Enhanced retinal ganglion cell (RGC) differentiation to organoid stages are demonstrated by expression in retinal neurons. Retinal organoids were differentiated using both traditional and standardized methods using a Brn3b-GFP reporter cell line to examine the first born retinal neurons. Using qPCR and mean fluorescent intensity quantifications of the RGC specific marker Brn3b, after 30 days of differentiation (FIGS. 5A-5B) the standardized retinal organoids robustly express Brn3b positive RGCs, while organoids differentiated using traditional methods have very low levels of Brn3b expression at Day 30 (FIGS. 5C, 5G). Retinal progenitor marker Chx10 staining shows RGCs develop faster when organoids are differentiated using the standardized method (FIG. 5F) compared to the traditional method (FIG. 5E). Using qPCR and mean fluorescent intensity quantifications of the cone-rod homeobox (CRX) marker after 60 days of differentiation, the standardized retinal organoids are robustly expressing CRX (FIGS. 5L, 5P), while organoids differentiated using traditional methods have low levels of CRX expression at Day 60 (FIGS. 5K, 5O). Retinal progenitor marker Chx10 staining shows nerve cells develop faster when organoids are differentiated using the standardized method (FIG. 5N) compared to the traditional method (FIG. 5M). Retinal organoid tissue differentiated using both traditional and standardized methods after 150 day of differentiation and formation (FIGS. 5Q, 5R) using a neural retina leucine zipper (NRL) marker, the cells are robustly expressing NRL positive (FIG. 5T), compared to traditional differentiated methods (FIG. 5S). NRL/ARR3/CRX staining shows differentiated retinal cell development using the standardized method (FIG. 5V) compared to the traditional method (FIG. 5U). NRL regulation of rhodopsin is also enhanced using standardized methods (FIG. 5X) compared to traditional methods (FIG. 5W).

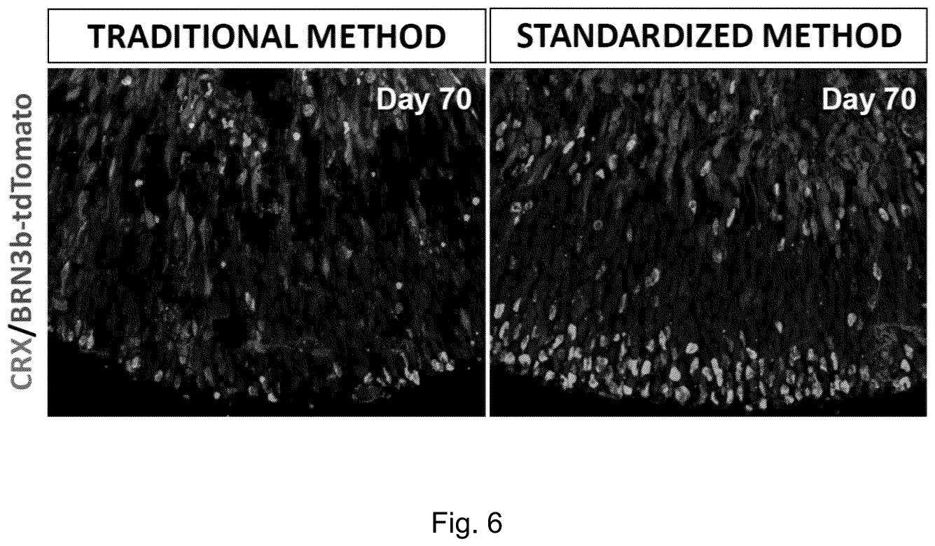

Expedited photoreceptor differentiation occurs at later stages of retinal organoid differentiation. Organoids were differentiated for 70 days using both the traditional and standardized methods and then analyzed for the expression of the photoreceptor specific marker CRX. Photoreceptor differentiation is expedited when retinal organoids are differentiated using standardized methods compared to traditional methods (FIG. 6).

A novel retinal organoid differentiation protocol has been presented using more standardized, rapid reaggregation methods to generate highly reproducible 3D retinal organoids from human pluripotent stem cells (hPSCs). BMP signaling contributing to retinal specification was analyzed by treatment with either BMP4 or the BMP inhibitor LDN-193189, and differentiation efficiency was assessed at various time points based on morphological analyses and the expression of retinal markers. Additionally, to identify transcriptional changes that underly retinal fate determination events, mRNA-seq analyses were conducted at the earliest stages of retinal specification.

As disclosed, retinal organoids generated using quick reaggregation methods were highly reproducible in both their size and shape compared to more traditional methods. Following treatment of early aggregates with either BMP4 or LDN-193189, pure populations of either retinal or forebrain organoids were derived, respectively. Subsequently, RNA-seq methods analyzed the transcriptional profile of the earlies stages of retinal vs forebrain specification, long before these lineages have been reliably identified previously. These refined methods also yielded retinal organoids with greatly expedited differentiation timelines, with differentiated retinal neurons arising at earlier stages than traditional differentiation methods, also exhibiting higher levels of self-organization.

Taken together, this disclosure provides a novel and highly reproducible method for generating retinal organoids from human pluripotent stem cells suitable for analyzing the earliest stages of human retinal fate specification in an organoid model. These results elucidate some of the earliest transcriptional changes occurring at the most immediate stages of human retinal development, and provide a more optimized and rapid method for generating retinal organoids for translational applications.

All publications, patents, and patent applications mentioned in this specification are herein incorporated by reference in their entirety as if each individual publication, patent, and patent application was specifically and individually indicated to be incorporated by reference.

1. A method of preparing pluripotent stem cells (PSCs) for use in retinal organoid differentiation, the method comprising:
(a) contacting the PSCs with accutase and a Rock inhibitor;
(b) dissociating the PSCs to single cells;
(c) culturing the dissociated cells in mTeSR for at least one day;
(d) culturing the cells of (c) in (1) mTeSR, and (2) neural induction medium, at a ratio of 3:1, for one day.

2. A method of preparing aggregates of pluripotent stem cells for use in differentiating three-dimensional retinal organoid tissue, the method comprising:
a) day 1: culture the cells of claim 1(d) in (1) mTeSR, and (2) Neural induction medium, at a ratio of 1:1, for one day;
b) day 2: culture the cells of (a) in (1) mTeSR and (2) Neural induction medium, at a ratio of 1:3 for 1 day;
c) day 3: culture the cells of (b) in Neural induction medium for three days, thereby forming aggregates.

3. A method of forming three-dimensional retinal organoid tissue, the method comprising:
a) day 6: culture the aggregates of claim 2 in Neural induction medium and BMP4 for two days, wherein the cells have formed into aggregates;
b) day 8: transfer 10-20 aggregates of (d) to a culture plate and culture the aggregates in the presence of reduced concentrations of BMP4 for eight days;
c) day 15: replace all media and culture aggregates in NIM, in the absence of FBS and culture for one day;
d) day 16: culture the aggregates of (f) in RDM supplemented with 1% FBS for six days; and
e) day 22: culture the aggregates of (g) in RDM with 10% FBS and 1× Glutamax for three days,
thereby forming three-dimensional retinal organoid tissue.

4. The method of claim 3, wherein the retinal organoid tissues expresses one or more retinal markers, or upregulated levels of one or more markers selected from SIX6, RAX, LHX9 VSX2 (CHX10), and PAX6.

5. The method of claim 3, further comprising:
f) day 25 culture the aggregates of (h) in RDM with 10% FBS, 1× Glutamax, and taurine for an additional 25 days.

6. The method of claim 5, further comprising:
g) day 50: culture the aggregates of (i) in RDM, 10% FBS, 1× Glutamax, taurine, retinoic acid, DHA, and BDNF, for an additional 40 days.

7. The method of claim 6, further comprising:
h) day 90; culture aggregates of (j) in RDM, 10% FBS, 1× Glutamax, taurine, DHA, and BDNF.

8. The method of claim 1, wherein the cells are human pluripotent stem cells (hPSCs).

9. The method of claim 8 wherein the hPSCs are induced hPSCs or embryonic hPSCs.

10. (canceled)

11. The method of claim 3, wherein the generated three-dimensional retinal organoid tissue comprises an improvement in size uniformity, and morphological uniformity, as compared to retinal organoid tissue prepared by alternative methods.

12. The method of claim 3, wherein the efficiency of retinal organoid tissue production is at least 80%, 90%, 95%, or 98% based on the number of aggregates at step 3(b).

13.-15. (canceled)

16. The method of claim 5, wherein after a total of 30 days of differentiation, starting at day 1, Brn3b is expressed at higher levels compared to retinal organoids prepared by alternative methods.

17. The method of claim 6, wherein after a total of 70 days of differentiation, starting at day 1, the photoreceptor specific marker CRX is expressed.

18. A method of obtaining three-dimensional human retinal organoid tissue, the method comprising:
a) day 1: culture cell aggregates in (1) mTeSR, and (2) Neural induction medium, at a ratio of 1:1, for one day;
b) day 2: culture the aggregates of (a) in (1) mTeSR and (2) Neural induction medium, at a ratio of 1:3 for 1 day;
c) day 3: culture the aggregates of (b) in Neural induction medium for three days;
d) day 6: culture the aggregates of (c) in Neural induction medium and BMP4 for two days;
e) day 8: transfer 10-20 aggregates of (d) to a single well of a 6-well culture plate and culture the aggregates and culture in the presence of reduced concentrations of BMP4 for eight days;
f) day 15: replace all media and culture aggregates in NIM, in the absence of FBS;
g) day 16: culture the aggregates of (f) in RDM supplemented with 1% FBS for six days;
h) day 22; culture the aggregates of (g) in RDM with 10% FBS and 1× Glutamax for three days;
i) day 25: culture the aggregates of (h) in RDM with 10% FBS, 1× Glutamax, and taurine for 25 days;
j) day 50: culture the aggregates of (i) in RDM, 10% FBS, 1× Glutamax, taurine, retinoic acid, DHA, and BDNF, for 40 days; and
k) day 90; culture aggregates of (j) in RDM, 10% FBS, 1× Glutamax, taurine, DHA, and BDNF,
thereby generating three-dimensional human retinal organoid tissue.

19. The method of claim 18, wherein the cell aggregates are derived from human pluripotent stem cells (hPSCs).

20. The method of claim 19, wherein the hPSCs are induced hPSCs or embryonic hPSCs.

21. (canceled)

22. The method of claim 18, wherein the generated three-dimensional retinal organoid tissue comprises an improvement in size uniformity and morphological uniformity as compared to retinal organoid tissue prepared by alternative methods.

23. The method of claim 18, wherein the efficiency of retinal organoid tissue production is at least 80%, 90%, 95%, or 98% based on the number of aggregates at step (e).

24.-26. (canceled)

27. The method of claim 22, wherein the generated three-dimensional human retinal organoids are more uniform in size and circularity as compared to organoids produced by alternative methods.

28. The method of claim 18, wherein the method does not include the addition of LDN.

29. The method of claim 18, wherein aggregates exhibit differentiated photoreceptors by day 70.
